(19) United States
(12) Patent Application Publication
(10) Pub. No.: US 2022/0401617 A1
(43) Pub. Date: Dec. 22, 2022

(54) ADHESIVE COMPOSITIONS COMPRISING THERAPEUTICS

(71) Applicant: RevBio, Inc., Lowell, MA (US)

(72) Inventors: Brian J. Hess, Charlestown, MA (US); Rahul P. Jadia, Lowell, MA (US); George W. Kay, Sharon, MA (US)

(21) Appl. No.: 17/774,409

(22) PCT Filed: Nov. 4, 2020
(86) PCT No.: PCT/US20/58945

§ 371 Date: May 4, 2022

Related U.S. Application Data
(60) Provisional application No. 62/930,467, filed on Nov. 4, 2019.

(51) Int. Cl. A61L 24/00

(52) U.S. Cl. CPC A61L 24/0063; A61L 24/0015; A61L 24/0036; A61L 24/0042; A61L 2300/428; A61L 2300/414; A61L 2430/02; A61L 2430/12; A61L 2300/442; A61L 2300/604; A61L 2300/402; A61L 2300/41; A61L 2300/406

(57) ABSTRACT

Compositions may include a therapeutic that is released from the composition to treat any number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof.

### FIELD OF TECHNOLOGY

Aspects and embodiments disclosed herein relate to adhesive compositions and methods of use thereof. More particularly, aspects and embodiments disclosed herein relate to adhesive compositions capable of joining two materials (for example, bone and bone, bone and tissue, or bone and device), occluding bone, filling bony defects, or comprising therapeutics for release or retention for use in medical applications.

### BACKGROUND

Bone regenerative biomaterials may be applied to defects of mineralized tissues in order to return the size, shape, contours, or continuity of the particular bone or bone aggregates to a better state of health. The defects might be a result of pathologic processes (e.g., a tumor, infection, arthritis of the spine or ankle, a cyst or a granuloma), trauma (e.g., simple or comminuted fracture), or osteotomies related to medical uses (e.g., cranioplasty, prosthetic knee joint replacement), or dental uses (e.g., removal of a wisdom tooth, removal of a cyst, or retrograde endodontic procedure). Procedures related to the surgical treatment of these defects are often followed by several days of post-operative pain, frequently requiring pain control measures. Post-operative pain is not just a result of tissue injury and damage, but may also result from exposure to noxious agents contributing to localized osteitis. These noxious agents may be microbes or foreign debris (e.g., shrapnel or dirt) present within or near the wound following surgical treatment (e.g., extraction of wisdom teeth, resection of a tumor, implant placement), biomaterials which create tissue damage or stimulate inflammatory response (e.g., polymethylmethacrylate-based bone cements), or damage related to heat exposure (e.g., from the traumatic event or from the osteotomy process itself). Post-operative pain following these procedures is often treated with systemic pain control agents which include opioids or opiates, as well as nonsteroidal anti-inflammatory drugs (NSAIDs), and combinations thereof.

The current standard of care for pain control involves the use of systemic analgesics, such as habit-forming opioids. The prescription of opioids for pain management has demonstrably placed many patients at increased risk of drug abuse and addiction. For example, just for wisdom tooth extraction, of which ten million are performed every year in the U.S alone, statistics show that there is a nearly 7% increase in persistent opioid use and roughly 6% increase in subsequent diagnosis of opioid abuse following a single prescription of opioid for post-operative pain. In addition to life threatening adverse reactions associated with intravenous opioid use, such as respiratory and cardiac arrest, there are a host of societal problems related to opioid addiction, which include crime, health care burden, loss of productivity, family and social distress, and others. As such, there exists a need for delivering non-habit-forming painkillers to patients, particularly in the dental field.

### SUMMARY

The present disclosure features a composition, such as an adhesive composition, useful for administering a therapeutic to a subject or sample, as well as related kits thereof. In some embodiments, the composition comprises a multivalent metal salt, a compound (e.g., a compound of Formula I, Formula II, Formula III, Formula IV, Formula V, or Formula VI or a combination thereof), a therapeutic, and an aqueous medium. In some embodiments, the multivalent metal salt comprises an alkaline earth element or a salt thereof. In some embodiments, the multivalent metal salt comprises a divalent metal, e.g., calcium. In some embodiments, the multivalent metal salt comprises tetracalcium phosphate or tricalcium phosphate (e.g., alpha tricalcium phosphate or beta tricalcium phosphate). In some embodiments, the compound of Formula I comprises phosphoserine. In some embodiments, the compound is present in an amount from about 10% to about 90% weight by weight (w/w) of the total composition. In some embodiments, the therapeutic is present in an amount from about 0.1% to about 40% weight by weight (w/w) of the total composition. In some embodiments, the composition comprises an adhesive strength upon curing of greater than 100 kPa (e.g., greater than 150 kPa, 200 kPa, 250 kPa, 300 kPa, 400 kPa, 500 kPa, 600 kPa, 750 kPa, 1000 kPa or more). In some embodiments, the composition further comprises an additive. In some embodiments, the composition is an adhesive composition and/or a bone regenerative composition.

In some embodiments, the additive comprises a salt, a filler, a formulation base, an abrasive, a coloring agent, a flavoring agent, a polymer, a viscosity modifier, a medication that acts locally, a medication that acts systemically, or an antimicrobial agent.

In some embodiments, the therapeutic comprises a pain reliever, an antibiotic, chemotherapeutic, hormone, protein, or contrast agent. In some embodiments, the pain reliever comprises an opioid, non-steroidal anti-inflammatory (NSAID), local anesthetic, or gabapentinoid. In some embodiments, the antibiotic comprises penicillin (β-lactams), a cephalosporin (γ-lactams), a macrolide, lincomycin, a nitroimidazole, a carbapenem, a fluoroquinolone, a sulfonamide, a tetracycline, an aminoglycoside, quinolone, a polyketide, or a glycopeptide. In some embodiments, the chemotherapeutic comprises an alkylating agent, an antimetabolite, an antibiotic, topoisomerase, or a tyrosine kinase inhibitor. In some embodiments, the hormone comprises a growth hormone or a cholecalciferol. In some embodiments, the therapeutic comprises a release rate to achieve sustained release until the composition partially or completely biodegrades. In some embodiments, the therapeutic may be retained within the composition for a prolonged time, e.g., scale of days, weeks, months, or years.

The present disclosure also features a kit comprising an adhesive composition comprising a multivalent metal salt, a compound (e.g., a compound of Formula (I), Formula (II), Formula (III), Formula (IV), Formula (V), Formula (VI), or a combination thereof), a therapeutic, and an aqueous medium. The kit may further comprise instructions of use. In some embodiments, the kit further comprises a device (e.g., bowl, spatula, syringe), or a plurality of devices, for preparation or application of the adhesive composition.

The present disclosure also features a method of using an adhesive composition comprising a therapeutic. The method may comprise applying the composition to a surgery site.

In an embodiment, the adhesive composition comprising a therapeutic disclosed herein may perform at least one of the following functions in addition to the adhesive bone regenerative (e.g., scaffold) functions later described in numerous embodiments: (i) serve as a vehicle for holding and gradually releasing a therapeutic (e.g., a pain control agent, an antibiotic, a hormone, a chemotactic agent, or another biologically active substance which might act locally or systemically); (ii) serve as a vehicle for holding a therapeutic agent (e.g., an antibiotic preventing adhesive composition colonization by pathogens, or another biologically active substance acting on the host or foreign biota); or (iii) occlude access to the bone or bone wound surface to undesirable factors (e.g., fibrous tissue, microbes, environmental debris such as decomposing food remnants, saliva, etc.) thereby preventing problems such as localized osteitis or osteomyelitis. In an embodiment, the composition comprises (i). In an embodiment, the composition comprises (ii). In an embodiment, the composition comprises (iii). In an embodiment, the composition comprises two of (i), (ii), and (iii). In an embodiment, the composition comprises (i) and (ii). In an embodiment, the composition comprises (ii) and (iii). In an embodiment, the composition comprises (i) and (iii). In an embodiment, the composition comprises each of (i), (ii), and (iii).

The composition comprises a therapeutic or a combination of therapeutics. For example, composition may comprise a therapeutic or a combination of therapeutics. The therapeutic may be selected to treat any one of a number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality, contrast medium), or any of a combination thereof. In some embodiments, the therapeutic used in the composition may mitigate post-operative pain.

In some embodiments, the therapeutic is a therapeutic or a combination thereof to treat pain (e.g., pain reliever). The pain reliever may include, but is not limited to, opioids, non-steroidal anti-inflammatory (NSAIDs), local anesthetics, or gabapentinoids. Opioids may include, but are not limited to, fentanyl, morphine, hydromorphone, oxycodone, hydrocodone, and codeine. NSAIDs (cyclooxygenase inhibitors) may include, but not are limited to, naproxen, ibuprofen, diclofenac and acetylsalicylic acid. Local anesthetics (sodium channel blockers) may include, but are not limited to, bupivacaine, lidocaine, prilocaine, and articaine. Gabapentinoids (calcium channel blockers) may include, but are not limited to, gabapentin, pregabalin, and mirogabalin.

In some embodiments, the therapeutic can be a therapeutic or a combination thereof to treat infection (e.g., an antibiotic). The antibiotics may include, but are not limited to, penicillins (β-lactams), cephalosporins (γ-lactams), macrolides, lincosamides, nitroimidazoles, carbapenems, fluoroquinolones, sulfonamides, tetracyclines, aminoglycosides, quinolones, fluoroquinolones, polyketides, and glycopeptides. Penicillins may include, but are not limited to, amoxicillin. Cephalosporins may include, but are not limited to, cefazolin, cefaclor, cephalexin ceftriaxone. Macrolides may include, but are not limited to, erythromycin, clarithromycin. Lincosamides may include, but are not limited to, lincomycin and clindamycin. Fluoroquinolones may include, but are not limited to, ciprofloxacin, ofloxacin. Sulfonamides may include, but not limited to, co-trimoxazole, and trimethoprim. Tetracyclines may include, but are not limited to, tetracycline, and doxycycline. Aminoglycosides may include, but are not limited to, gentamicin, and tobramycin. Glycopeptides may include, but are not limited to, vancomycin, teicoplanin, telavancin, ramoplanin, decaplanin, and bleomycin.

In some embodiments, the therapeutic can be a therapeutic or a combination thereof to treat cancer (e.g., chemotherapeutic). Chemotherapeutic agents may include, but are not limited to, alkylating agents, antimetabolites, antibiotics, and topoisomerase and tyrosine kinase inhibitors. The alkylating agents may include, but are not limited to, cyclophosphamide, dacarbazine, temozolomide, and aziridine. Antimetabolites may include, but are not limited to, 5-Fluorouracil, cytarabine, gemcitabine, and methotrexate. Anticancer antibiotics may include, but are not limited to epirubicin, and idarubicin. Topoisomerase inhibitors may include, but are not limited to, irinotecan, camptothecin, doxorubicin, daunorubicin, and teniposide. Tyrosine kinase inhibitor may include, but is not limited to, erlotinib, sunitinib, dasatinib, and axitinib.

In some embodiments, the therapeutic can be a therapeutic or a combination thereof to regenerate tissue (e.g., growth hormone, bone morphogenetic protein, and hormones). Bone growth adjuvants may include acidic compounds, such as citric acid. Hormones may include, but are not limited to, the anabolic steroids, e.g., estrogen, testosterone, etc., and calciferols, e.g., cholecalciferol, i.e., Vitamin D₃, ergocalciferol, i.e., Vitamin D₂, and others.

In some embodiments, the therapeutic can be a therapeutic or a combination thereof to enhance imaging. Imaging agents may include, but are not limited to, gadolinium-based contrast agent, barium-based contrast agents, and iron/iron oxide-based contrast agent.

In some embodiments, the foregoing therapeutics may be loaded singly in a monotherapy approach, or in multiples in a combination therapy. The therapeutics loaded into the compositions may also be used as sole source of the therapeutic or in combination with therapeutics and therapies, administered by other routes (e.g., topical, oral, intramuscular, subcutaneous, depot injection, implant seed, etc.).

In some embodiments, the composition disclosed herein exhibits adhesive behavior toward bone and other materials, including titanium and stainless steel. In some embodiments, the composition disclosed herein exhibits bone regenerative behavior, wherein the self-setting composition is gradually resorbed and replaced with new bone in volume-maintaining manner over time. The composition may also exhibit high adhesive strength (e.g., greater than 100 kPa, e.g., greater than 150 kPa, 200 kPa, 250 kPa, 300 kPa, 400 kPa, 500 kPa, 600 kPa, 750 kPa, 1000 kPa or more). The adhesive composition may further comprise a therapeutic (e.g., pain reliever, antibiotic, anticlastic drug, growth hormone, bone morphogenetic protein, chemotherapeutic, imaging modality) for which the bone regenerative composition occludes access to the bone or bone wound surface to undesirable factors, or for which the therapeutic is either retained or locally and controllably released from the composition to treat any one of the following ailments or conditions (e.g., pain, infection, cancer, osteoporosis), or to help accelerate local tissue regeneration (e.g., growth hormone, bone morphogenic protein), or to assist with surgical or therapeutic treatment (e.g., imaging modality), or any of a combination thereof. In some embodiments, the therapeutic used in the composition mitigates post-operative pain (e.g., reduces post-operative pain).

Exemplary multivalent metal salts may be organic or inorganic in nature and may include calcium phosphates (e.g., hydroxyapatite, octacalcium phosphate, tetracalcium phosphate, tricalcium phosphate), calcium nitrate, calcium citrate, sodium citrate, calcium carbonate, magnesium phosphates, sodium silicates, lithium phosphates, titanium phosphates, strontium phosphates, barium phosphates, zinc phosphates, calcium oxide, magnesium oxide, barium sulfate and combinations thereof. Exemplary multivalent metal salts may be a salt of an alkaline earth element (e.g., beryllium, magnesium, barium, radium, strontium, or calcium).

In an aspect, provided herein are adhesive compositions comprising a multivalent metal salt (e.g., calcium), a compound of Formula (I), a therapeutic and an aqueous medium, wherein the compound of Formula (I) is:

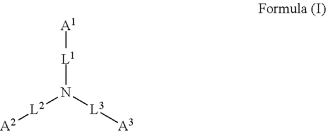

or a salt thereof, wherein: each of A¹, A², and A³ is independently selected from an acidic group (e.g., a carboxyl or phosphonyl); and each of L¹, L², and L³ is independently bond, alkylene (e.g., C₁-C₆ alkylene), or heteroalkylene (e.g., C₁-C₆ heteroalkylene).

In some embodiments, each of A¹, A², and A³ is independently a carboxyl or phosphonyl. In some embodiments, A¹ is carboxyl, and A² and A³ are phosphonyl. In some embodiments, A¹, A² and A³ are phosphonyl.

In some embodiments, each of L¹, L², and L³ is C₁-C₃ alkylene. In some embodiments, each of L¹, L², and L³ is C₁ alkylene.

In some embodiments, the compound of Formula (I) is a compound of Formula (I-a) or (I-b):

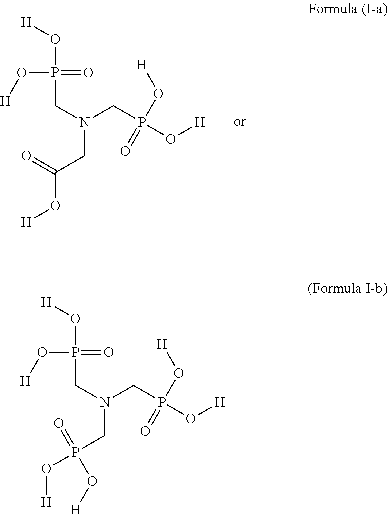 [SMILES: O=C(O)CN(CP(=O)(O)O)CP(=O)(O)O ; O=P(O)(O)CN(CP(=O)(O)O)CP(=O)(O)O]

In some embodiments, the aqueous medium is water. In some embodiments, the composition further comprises an additive.

In another aspect, provided herein are adhesive compositions comprising a multivalent metal salt (e.g., calcium), a compound of Formula (II), a therapeutic and an aqueous medium, wherein the compound of Formula (II) is:

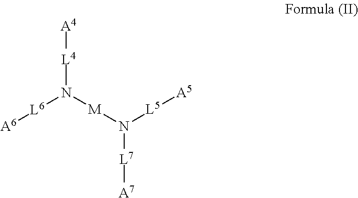

or a salt thereof, wherein: each of A⁴, A⁵, and A⁶, is independently selected from an acidic group (e.g., a carboxyl or phosphonyl); A⁷ is selected from an acidic group (e.g., a carboxyl or phosphonyl), a hydrogen atom, an alkyl, an aryl, a hydroxy group, a thio group, and an amino group; each of L⁴, L⁵, L⁶, and L⁷ is independently bond, alkylene (e.g., C₁-C₆ alkylene), or heteroalkylene (e.g., C₁-C₆ heteroalkylene); and M is alkylene (e.g., C₁-C₆ alkylene) or heteroalkylene (e.g., C₁-C₆ heteroalkylene).

In some embodiments, A⁴, A⁵, A⁶ and A⁷ are carboxyl.

In some embodiments, L⁴, L⁵, L⁶, and L⁷ are C₁-C₃ alkylene. In some embodiments, L⁴, L⁵, L⁶, and L⁷ are C₁ alkylene.

In some embodiments, M is C₁-C₄ alkylene. In some embodiments, M is C₂ alkylene. In some embodiments, M is C₃ alkylene. In some embodiments, M is C₁-C₆ heteroalkylene. In some embodiments, M is C₆ heteroalkylene. In some embodiments, M is bis(ethyleneoxy)ethylene. In some embodiments, M includes side chains. In some embodiments, M includes multiple side chains. In some embodiments, M includes one or multiple carboxymethylene side chains. In some embodiments, M includes one or multiple N-carboxymethylene groups or N-hydroxymethylene groups.

In some embodiments, the compound of Formula (II) includes three, four, five, six, or more N-carboxymethylene groups. In some embodiments, the compound of Formula (II) comprises ethylenediamine tetraacetic acid (EDTA). In some embodiments, the compound of Formula (II) is a compound of Formula (II-a), (II-b), (II-c), (II-d), (II-e), or (II-f):

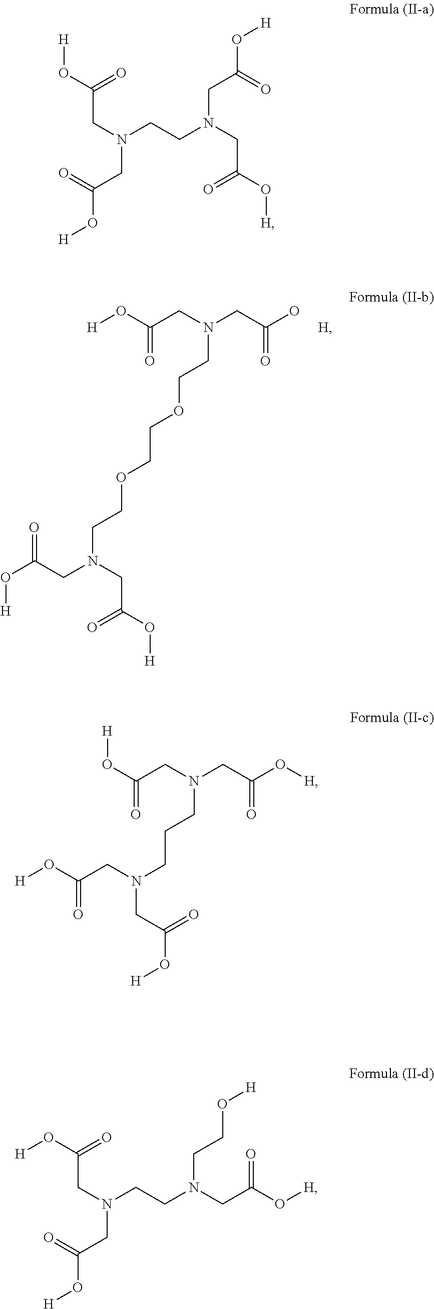

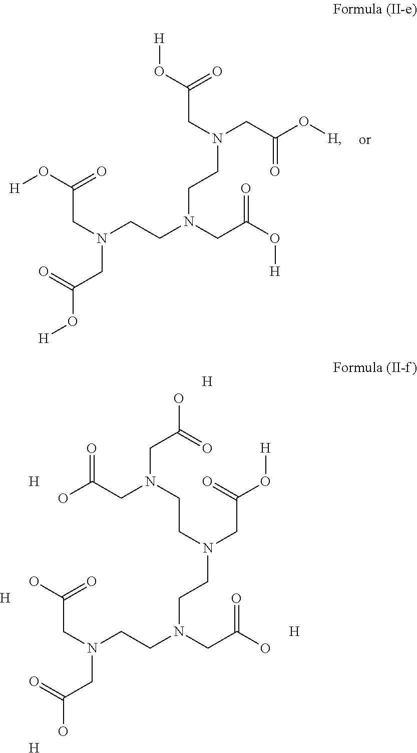

In some embodiments, the aqueous medium is water. In some embodiments, the composition further comprises an additive.

In another aspect, provided herein is an adhesive composition comprising a multivalent metal salt (e.g., calcium), a compound of Formula (III), a therapeutic and an aqueous medium, wherein the compound of Formula (III) is:

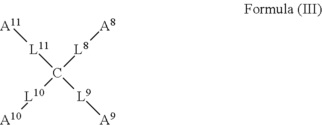

or a salt thereof, wherein: each of A⁸ and A⁹ is independently selected from an acidic group (e.g., a carboxyl or phosphonyl); each of A¹⁰ and A¹¹ is independently selected from an acidic group (e.g., a carboxyl or phosphonyl), a hydrogen atom, an alkyl, aryl, a hydroxy group, a thio group, and an amino group; each of L⁸, L⁹, L¹⁰ and L¹¹ is independently bond, alkylene (e.g., C₁-C₆ alkylene), or heteroalkylene (e.g., C₁-C₆ heteroalkylene.

In some embodiments, A⁸, A⁹, and A¹⁰ are carboxyl. In some embodiments, A¹⁰, A¹¹, are a hydrogen atom. In some embodiments, A¹¹ is a hydroxy or amino group. In some embodiments, L⁸, L⁹, L¹⁰, and L¹¹ are a bond. In some embodiments, L⁸ and L⁹ are C₁-C₃ alkylene. In some embodiments L¹¹ is a heteroalkylene (e.g., C₁-C₆ heteroalkylene). In some embodiments L¹¹ is methylenethiomethylene. In some embodiments, the compound of Formula (III) comprises citric acid or malonic acid. In some embodiments, the compound of Formula (III) is a compound of Formula (III-a), (III-b), (III-c), or (III-d):

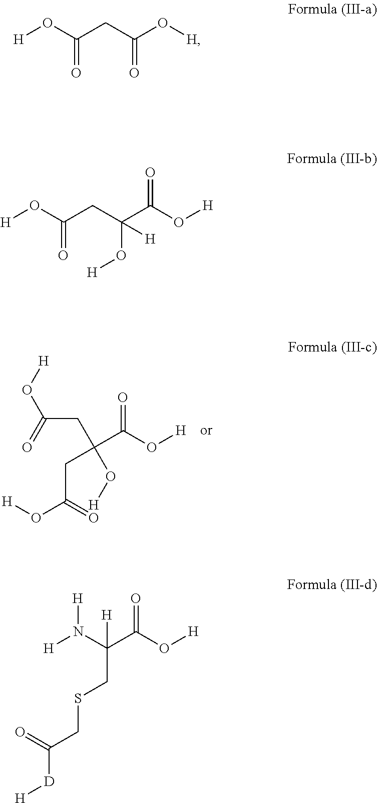

In some embodiments, the aqueous medium is water. In some embodiments, the composition further comprises an additive.

In yet another aspect, provided herein is an adhesive composition comprising a multivalent metal salt (e.g., calcium), a compound of Formula (IV), a therapeutic and an aqueous medium, wherein the compound of Formula (IV) is:

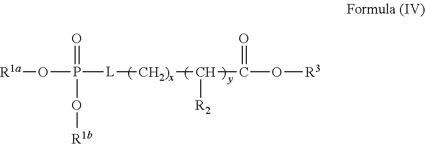

or a salt thereof, wherein: L is O, S, NH, or CH₂; each of R^{1a} and R^{1b} is independently H, an optionally substituted alkyl, or an optionally substituted aryl; R² is H, NR^{4a}R^{4b}, C(O)R⁵, or C(O)OR⁵; R³ is H, an optionally substituted alkyl, or an optionally substituted aryl; each of R^{4a} and R^{4b} is independently H, C(O)R⁶, or an optionally substituted alkyl; R⁵ is H, an optionally substituted alkyl, or an optionally substituted aryl; R⁶ is an optionally substituted alkyl or an optionally substituted aryl; and each of x and y is independently 0, 1, 2, or 3.

In some embodiments, L is O or S. In some embodiments, L is O. In some embodiments, each of R^{1a} and R^{1b} is independently H. In some embodiments, L is O, and each of R^{1a} and R^{1b} is H.

In some embodiments, R² is selected from H, NR^{4a}R^{4b}, and C(O)R⁵. In some embodiments, R² is NR^{4a}R^{4b}. In some embodiments, R² is NR^{4a}R^{4b} and each of R^{4a} and R^{4b} is independently H.

In some embodiments, L is O, each of R^{1a} and R^{1b} is independently H, R² is NR^{4a}R^{4b}, and each of R^{4a} and R^{4b} is independently H.

In some embodiments, R³ is H. In some embodiments, L is O, each of R^{1a} and R^{1b} is independently H, R² is NR^{4a}R^{4b}, each of R^{4a} and R^{4b} is independently H, and R³ is H.

In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1.

In some embodiments, the compound of Formula (IV) is phosphoserine. In some embodiments, the aqueous medium is water.

In another aspect, provided herein is an adhesive composition comprising a multivalent metal salt (e.g., calcium), a compound of Formula (V), a therapeutic and an aqueous medium, wherein the compound of Formula (V) is:

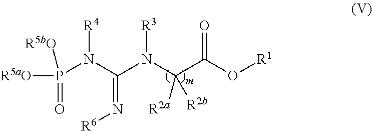

or a salt thereof, wherein R¹ is H, optionally substituted alkyl, optionally substituted alkenyl, optionally substituted alkynyl, optionally substituted aryl, or optionally substituted heteroaryl; each of R^{2a} and R^{2b} is independently H, optionally substituted alkyl, hydroxy, alkoxy, or halo; each of R³ and R⁴ is independently H or optionally substituted alkyl; each of R^{5a} and R^{5b} is independently H, optionally substituted alkyl, optionally substituted alkenyl, optionally substituted alkynyl, optionally substituted aryl, or optionally substituted heteroaryl; R⁶ is H or optionally substituted alkyl; and m is 1, 2, 3, 4, or 5.

In some embodiments, R¹ is H. In some embodiments, each of R^{2a} and R^{2b} is independently H. In some embodiments, m is 1. In some embodiments, each of R³ and R⁴ is H. In some embodiments, each of R^{5a} and R^{5b} is independently H. In some embodiments, R6 is H. In some embodiments, the compound of Formula (V) is a phosphocreatine. In some embodiments, the compound of Formula (V) is Formula (V-a):

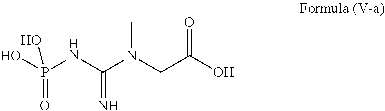

In some embodiments, the compound of Formula (V) is phosphocreatine (e.g., Formula (V-a). In some embodiments, the aqueous medium is water.

In another aspect, provided herein is a composition comprising a multivalent metal salt (e.g., calcium), a compound of Formula (VI), a therapeutic and an aqueous medium, wherein the compound of Formula (VI) is a nucleoside or nucleotide. In some embodiments, the compound of Formula (VI) is:

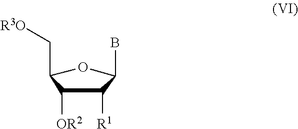

or a salt thereof, wherein B is a nucleobase; R¹ is H, OR⁴, or halo; R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl; R³ is H, optionally substituted alkyl, or a phosphate moiety (e.g., monophosphate or diphosphate); and R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl.

In some embodiments, B is a naturally occurring nucleobase or a non-naturally occurring nucleobase. In some embodiments, B comprises adenine, cytosine, guanosine, thymine, or uracil. In some embodiments, each of R¹, R², and R³ is H. In some embodiments, R3 is a phosphate group, e.g., a monophosphate, diphosphate, or triphosphate. In some embodiments, the compound of Formula (VI) is Formula (VI-a) or (VI-b):

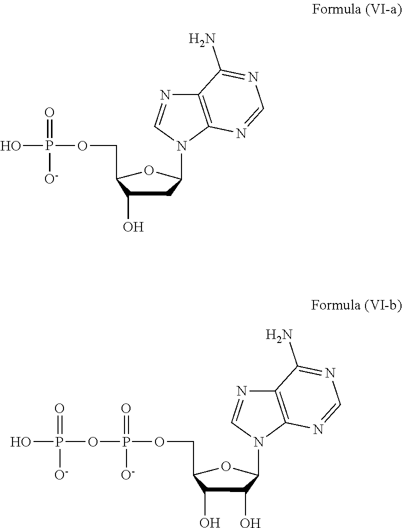

For example, the compound of Formula (VI) is 2′-deoxyadenosine monophosphate or 2′-deoxyadenosine diphosphate. In some embodiments, the aqueous medium is water.

Compositions including additives are disclosed herein. The compositions described herein comprising an additive may be an adhesive composition or a bone regenerative composition. Other embodiments are within the scope of the disclosure.

In some embodiments, the additive may comprise a salt (e.g., calcium carbonate, calcium bicarbonate, sodium carbonate, sodium bicarbonate, sodium chloride, potassium chloride). The additive may comprise one or more of a filler, a formulation base, an abrasive (e.g., bone fragment), a coloring agent (e.g., dye, pigment, or opacifier), a flavoring agent (e.g., sweetener), or a polymer. The additive may comprise a viscosity modifier (e.g., a polyol, a saccharide or polysaccharide (e.g., glycerol, mannitol, sorbitol, trehalose, lactose, glucose, fructose, sucrose, or a starch), or branched polyethylene glycol). The additive may comprise a medication that acts locally (e.g., anesthetic, coagulant, clotting factor, chemotactic agent, and agent inducing phenotypic change in local cells or tissues), a signaling factor or agent inducing phenotypic change in local cells or tissues), a hormone (e.g., endocrine, paracrine, or juxtacrine), a medication that acts systemically (e.g., analgesic, anticoagulant, enzyme co-factor, vitamin, pain reliever, anti-inflammatory agent, chemotactic agent, or an antimicrobial agent (e.g., antibacterial, antiviral, or antifungal agent).

In some embodiments, the compound is present in an amount from about 10% to about 90% weight by weight (w/w) of the total composition.

In some embodiments, the compound of a Formula (e.g., Formula (I), Formula (II), Formula (III), Formula (IV), Formula (V), Formula (VI), or a combination thereof), is initially provided as a powder and has a mean particle size of less than about 1.000 mm.

In some embodiments, the multivalent metal salt is initially provided as a powder and has a mean particle size of less than about 1.000 mm.

In some embodiments, the viscosity of the adhesive composition when in its fluid state might be as low as about 100 cP to about 10,000 cP and in its semi-solid state from about 10,000 cP to about 250,000 cP. In some embodiments, the viscosity and cohesion properties of the composition may facilitate the ability to squeeze the material through a needle or cannula as small as 18 gauge when the viscosity is in the low range of its fluid state.

In some embodiments, the composition has a tacky state for up to 12 minutes after mixing with the aqueous medium. During the tacky state, the composition may have a tack stress in the range of about 10 kPa to about 250 kPa after mixing with the aqueous medium.

In some embodiments, the composition has a putty state for up to 15 minutes after mixing with the aqueous medium. During the putty state, the composition may have a tack stress in the range of about 10 kPa to about 250 kPa after mixing with the aqueous medium.

In some embodiments, the composition has an adhesive strength upon curing of greater than 100 kPa.

In some embodiments, the composition comprises discrete, solid particles surrounded by a tacky, viscous liquid.

In some embodiments, the composition is a solid and comprises a therapeutic.

In some embodiments, the composition is a liquid and comprises a therapeutic.

In some embodiments, a plurality of therapeutics might be comprised within the composition.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic drawing of a gap, hole, or void on a surface.

FIG. 2 is a schematic drawing of a structure placed within the gap, hole, or void shown in FIG. 1.

FIG. 3 is a schematic drawing of an adhesive composition, optionally comprising a therapeutic, used to fill a gap, hole, or void on a surface after placement of a structure, as shown in FIGS. 1 and 2, for which the adhesive composition occludes access to the bone or bone wound surface to undesirable factors or for which the therapeutic is either retained or locally and controllably released from the composition, according to an embodiment disclosed herein.

FIG. 4 is a schematic drawing of a gap, hole, or void on a surface.

FIG. 5 is a schematic drawing of an adhesive composition, optionally comprising a therapeutic, used to fill a gap, hole, or void on the surface shown in FIG. 4, for which the adhesive composition occludes access to the bone or bone wound surface to undesirable factors or for which the therapeutic is either retained or locally and controllably released from the composition, according to an embodiment disclosed herein.

FIG. 6 is a schematic drawing of a structure placed within the gap, hole, or void on the surface shown in FIG. 5 having been filled with an adhesive composition, optionally comprising a therapeutic, for which the adhesive composition occludes access to the bone or bone wound surface to undesirable factors or for which the therapeutic is either retained or locally and controllably released from the composition, according to an embodiment disclosed herein;

FIG. 7 is an alternate schematic drawing of a gap, hole, or void on a surface.

FIG. 8 is a schematic drawing of an adhesive composition, optionally comprising a therapeutic, used to fill a gap, hole, or void on the surface shown in FIG. 7, for which the adhesive composition occludes access to the bone or bone wound surface to undesirable factors or for which the therapeutic is either retained or locally and controllably released from the composition, according to an embodiment disclosed herein.

FIG. 9 is a schematic drawing of a structure placed within the gap, hole, or void on the surface shown in FIG. 8 having been filled with an adhesive for which the bone regenerative composition, optionally comprising a therapeutic, occludes access to the bone or bone wound surface to undesirable factors or for which the therapeutic is either retained or locally and controllably released from the composition, according to an embodiment disclosed herein;

FIG. 10 is an alternate schematic drawing of a gap, hole, or void on a surface.

FIG. 11 is a schematic drawing of an adhesive composition, optionally comprising a therapeutic, used to fill a gap, hole, or void on the surface shown in FIG. 10, for which the bone regenerative composition occludes access to the bone or bone wound surface to undesirable factors or for which the therapeutic is either retained or locally and controllably released from the composition, according to an embodiment disclosed herein.

FIG. 12 is a schematic drawing of a structure placed within the gap, hole, or void on the surface shown in FIG. 11 having been filled with an adhesive composition, optionally comprising a therapeutic, occludes access to the bone or bone wound surface to undesirable factors or for which the therapeutic is either retained or locally and controllably released from the composition, according to an embodiment disclosed herein.

FIG. 13 is an alternate schematic drawing of a gap, hole, or void on a surface.

FIG. 14 is a schematic drawing of an adhesive composition, optionally comprising a therapeutic, used to fill a gap, hole, or void on the surface shown in FIG. 13, for which the bone regenerative composition occludes access to the bone or bone wound surface to undesirable factors or for which the therapeutic is either retained or locally and controllably released from the composition, according to an embodiment disclosed herein.

FIG. 15 is a schematic drawing of a structure placed within the gap, hole, or void on the surface shown in FIG. 14 having been filled with an adhesive composition, optionally comprising a therapeutic, for which the adhesive composition occludes access to the bone or bone wound surface to undesirable factors or for which the therapeutic is either retained or locally and controllably released from the composition, according to an embodiment disclosed herein.

FIG. 16 is a schematic drawing of an adhesive composition, optionally comprising a therapeutic, being delivered to an application site via injection, for which the adhesive composition occludes access to the bone or bone wound surface to undesirable factors or for which the therapeutic is either retained or locally and controllably released from the composition, according to an embodiment disclosed herein.

FIG. 17 is an alternate schematic drawing of an adhesive composition, optionally comprising a therapeutic, being delivered to an application site via injection, for which the bone regenerative composition occludes access to the bone or bone wound surface to undesirable factors or for which the therapeutic is either retained or locally and controllably released from the composition, according to an embodiment disclosed herein.

FIG. 18 is an alternate schematic drawing of an adhesive composition, optionally comprising a therapeutic, being delivered to an application site via injection, for which the bone regenerative composition occludes access to the bone or bone wound surface to undesirable factors or for which the therapeutic is either retained or locally and controllably released from the composition, according to an embodiment disclosed herein.

FIG. 19 is an alternate schematic drawing of an adhesive composition, optionally comprising a therapeutic, being delivered to an application site via injection, for which the bone regenerative composition occludes access to the bone or bone wound surface to undesirable factors or for which the therapeutic is either retained or locally and controllably released from the composition, according to an embodiment disclosed herein.

FIG. 20 is an alternate schematic drawing of an adhesive composition, optionally comprising a therapeutic, being delivered to an application site via injection, for which the bone regenerative composition occludes access to the bone or bone wound surface to undesirable factors or for which the therapeutic is either retained or locally and controllably released from the composition, according to an embodiment disclosed herein.

FIG. 21 is a schematic drawing of an adhesive composition, optionally comprising a therapeutic, used to create a seal, for example by filling a gap, hole, or void, in a structure application site, for which the bone regenerative composition occludes access to the bone or bone wound surface to undesirable factors or for which the therapeutic is either retained or locally and controllably released from the composition, according to an embodiment disclosed herein.

FIG. 22 is an alternate schematic drawing of an adhesive composition, optionally comprising a therapeutic, used to create a seal, for example by filling a gap, hole, or void, in a structure application site, for which the bone regenerative composition occludes access to the bone or bone wound surface to undesirable factors or for which the therapeutic is either retained or locally and controllably released from the composition, according to an embodiment disclosed herein.

FIG. 23 is an overlay of solid-state NMR spectra showing the following: i) an exemplary adhesive composition comprising tetracalcium phosphate and phosphoserine (101); ii) naproxen (102); iii) the adhesive composition of i) plus naproxen before incubation (103); and iv) the adhesive composition of i) plus naproxen after incubation (104). This testing shows that naproxen was almost fully eluted from the adhesive composition with no modification of the naproxen chemical structure upon inclusion in the adhesive composition.

FIG. 24 is a graph depicting an elution profile of an exemplary therapeutic agent (gentamicin) in 1× PBS of 2.5% AB w/w dose (201) and 5.0% AB w/w dose (202).

FIG. 25 shows the % drug release of various adhesive composition-drug formulations after the incubation period, e.g., naproxen in the composition, diclofenac in the composition, bupivacaine in the composition, and articaine in the composition.

FIG. 26 is a plot that compares % bupivacaine release between porous adhesive compositions formulation and non-porous adhesive compositions formulation after the incubation period. This HPLC data confirms that at 7-day period, the porous formulation showed remarkable increase in the % release of the drug compared to the non-porous formulation.

### DETAILED DESCRIPTION

The present disclosure features compositions and methods of use thereof which comprises a therapeutic, e.g., a NSAID. In an embodiment, the compositions described herein exhibit the advantageous property of robust adhesive behavior toward bone and other materials, (e.g., titanium or other metals), followed by bone regenerative behavior wherein the self-setting adhesive composition is gradually resorbed and replaced with new volume maintaining bone over time. In some embodiments, the adhesive composition may be used to occlude access to the bone or bone wound surface to undesirable factors. The therapeutic may be a pain reliever, antibiotic, anticlastic drug, growth hormone, bone morphogenetic protein, chemotherapeutic, imaging modality, which, in some embodiments, may be loaded into the adhesive composition for retained use (e.g., prevention of bacterial colonization or infection into or onto the material) or for localized and controlled release to treat a condition (e.g., pain, infection, cancer, osteoporosis).

In some embodiments, the composition may be used for pain control. The therapeutic may be locally and controllably released. Exemplary uses of the composition described herein include treatment of a contaminated wound near a bone tissue, prevention of a post-operative infection of tissues in the vicinity of the deposited adhesive composition, or colonization of the surface or the interior of the substance of the adhesive composition, treatment to kill residual tumor cells present in the local bone tissue following tumor resection from bone, or treatment to address osteoporosis to prevent bone loss. In some embodiments, the adhesive composition comprises a therapeutic as described herein for local and controllable rate of release.

In some embodiments, the adhesive composition features an interaction between reagents which are electrically charged, but not purely anionic or purely cationic (e.g., zwitterion) and a mineral salt of a multivalent metal (e.g., tetra-calcium phosphate). For example, the interaction may be an acid base reaction resulting in the partial or complete consumption of the original mineral reagent, e.g., tetra-calcium phosphate, and the precipitation of a new distinct mineral phase, e.g., hydroxyapatite, calcium phosphoserine monohydrate. In some embodiments, the structure of the anionic components disclosed herein may also involve multiple nucleophilic groups which chelate or coordinate around the mineral multivalent metal subcomponents.

In some embodiments, the adhesive composition is self-setting. For example, the interaction of the components of the composition may result in the production of a tacky and adhesive reaction mixture. In particular, the interaction may result in a viscous liquid which then solidifies forming a persistent bond to high-energy surfaces, e.g., bone, metal, glass, etc. In some embodiments, the adhesive bond interaction between the adhesive composition and the substrate surface may occur whether the substrate is dry or wet (e.g., dampened or submerged in an aqueous medium). In some embodiments, after the adhesive composition has been implanted, the therapeutic or combination thereof is released, for either retained use, or local and controllable rate of release. In some embodiments, either retained use or localized and controllable rate of release of said therapeutic to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein.

“Alkyl” refers to a radical of a straight-chain or branched saturated hydrocarbon group. In some embodiments, an alkyl group has 1 to 3 carbon atoms (“C₁₋₃ alkyl”). In some embodiments, an alkyl group has 1 carbon atom (“C₁ alkyl”). Examples of C₁₋₆ alkyl groups include methyl (C₁) and propyl (C₃). Alkyl groups disclosed herein may be substituted or unsubstituted.

As used herein, “alkylene,” refers to a divalent radical of an alkyl group. When a range or number of carbons is provided for a particular “alkylene” group, it is understood that the range or number refers to the range or number of carbons in the linear carbon divalent chain.

The term “heteroalkyl,” as used herein, refers to an alkyl group, as defined herein, which further comprises 1 or more (e.g., 1 or 2) heteroatoms (e.g., non-ionizable heteroatoms, e.g., oxygen) within the parent chain, wherein the one or more heteroatoms is inserted between adjacent carbon atoms within the parent carbon chain and/or one or more heteroatoms is inserted between a carbon atom and the parent molecule, i.e., between the point of attachment.

As used herein, “alkylene,” and “heteroalkylene,” refer to a divalent radical of an alkyl and heteroalkyl group respectively. When a range or number of carbons is provided for a particular “alkylene” or “heteroalkylene” group, it is understood that the range or number refers to the range or number of carbons in the linear carbon divalent chain.

As used herein, “carboxyl” refers to —C(O)OH.

As used herein, “phosphonyl” refers to —P(O)(OH)₂.

As used herein, “aryl” refers to a functional group or substituent derived from an aromatic ring. In some embodiments, an aryl may be derived from an aromatic hydrocarbon. Exemplary aryl groups include, but are not limited to, phenyl, naphthyl, thienyl, indolyl, and xylyl. Aryl groups disclosed herein may be substituted or unsubstituted.

As used herein, “hydroxy” refers to —OH.

As used herein “thiol” refers to a sulfur analog of an alcohol. In some embodiments, a thiol group may include an organosulfur compound, for example, one that contains a carbon-bonded sulfhydryl. Exemplary thiol groups include —SH, —C—SH, and R—SH, where R represents an organic substituent, e.g. an aryl or alkyl.

As used herein “amino” refers to a compound that contains a nitrogen atom, for example, with a lone pair, attached to a hydrogen atom, alkyl group, or aryl group. In some embodiments, the amino may be derived from ammonia, for example, wherein one or more hydrogen atoms have been replaced by a substituent, for example an aryl or alkyl. The amino may be organic or inorganic. In some embodiments, an amino includes —NH₂, an amino acid, a biogenic amine, trimethylamine, and aniline.

### Compositions

The present disclosure features compositions described herein including adhesive, self-setting compositions comprising a mixture of a multivalent metal salt, e.g., tetra-calcium phosphate, a compound of a Formula (e.g., Formula (I), Formula (II), Formula (III), Formula (IV), Formula (V), Formula (VI), or a combination thereof), and a therapeutic or a combination thereof (e.g., pain reliever, antibiotic, anticlastic drug, growth hormone, bone morphogenetic protein, chemotherapeutic, imaging modality), and an aqueous medium, e.g., water.

Exemplary multivalent metal salts may be organic or inorganic in nature and include calcium phosphates (e.g., hydroxyapatite, octacalcium phosphate, tetra-calcium phosphate, tricalcium phosphate), calcium nitrate, calcium citrate, sodium citrate, calcium carbonate, magnesium phosphates, magnesium citrate, strontium citrate, sodium silicates, lithium phosphates, titanium phosphates, strontium phosphates, barium phosphates, zinc phosphates, calcium oxide, magnesium oxide, and combinations thereof.

The amount of each multivalent metal salt (e.g., a calcium phosphate or calcium oxide or a combination thereof) may vary, e.g., between about 10% to about 90 weight by weight (w/w) of the total composition. In some embodiments, the amount of the multivalent metal salt (e.g., a calcium phosphate or calcium oxide or a combination thereof) is in the range of about 10% to about 90%, about 15% to about 85%, about 20% to about 80%, about 30% to about 75%, about 40% to about 70%, or about 50% to about 65% w/w of the total composition. In other embodiments, the amount of the metal salt (e.g., a calcium phosphate or calcium oxide or a combination thereof) is in the range of about 5% to about 95%, about 10% to about 85%, about 15% to about 75%, about 20% to about 65%, about 25% to about 55%, or about 35% to about 50% w/w of the total composition.

In some embodiments, the multivalent metal salt comprises one or more alkaline earth metals, e.g., beryllium, magnesium, barium, radium, strontium, or calcium. In some embodiments, the multivalent metal salt may comprise a mixed salt of several metal ions, e.g., a mixed salt of alkali earth metal ions. In some embodiments, the multivalent metal salt comprises calcium. In some embodiments, the multivalent metal salt comprises calcium and phosphate. In some embodiments, the multivalent metal salt comprises tetra-calcium phosphate. In some embodiments, the composition comprises a plurality of multivalent metal salt compounds. In some embodiments, the plurality comprises tetra-calcium phosphate and at least one other multivalent metal salt compound. In some embodiments, the multivalent metal salt comprises hydroxyapatite. In some embodiments, the multivalent metal salts comprise tricalcium phosphate. In some embodiments, the tricalcium phosphate comprises either alpha tricalcium phosphate or beta tricalcium phosphate. In some embodiments, the multivalent metal salts comprise an oxide. In some embodiments, the multivalent metal salt is calcium oxide. In some embodiments, the multivalent metal salt compound does not comprise tetra-calcium phosphate. In some embodiments, the composition comprises tricalcium phosphate and calcium oxide.

In some embodiments, the multivalent metal salt is initially provided as a powder or as a granule. These powders may exhibit a mean particle size of about 0.0001 to about 1.000 mm, about 0.0005 to about 0.001 mm, about 0.001 to about 0.025 mm, about 0.005 to about 0.015 mm, about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The powder may have a mean particle size of less than about 1.000 mm. The particle size distribution may be multi-modal to include any combination of mean particle sizes as previously described. These granules may exhibit a mean granule size of about 0.050 mm to about 5 mm, about 0.100 to about 1.500 mm, about 0.125 to about 1.000 mm, about 0.125 to about 0.500 mm, about 0.125 to about 0.250 mm, about 0.250 to about 0.750 mm, about 0.250 to about 0.500 mm, about 0.500 to about 1.00 mm, about 0.500 to about 0.750 mm. The granule size distribution may be multi-modal to include any combination of mean granule sizes as previously described. In some embodiments, the granules may be supplied with a various proportion of porosity and a various size of internal pores. The pores may communicate with each other. The pores may communicate with granule surface. In some embodiments, the pores do not communicate with each other. In some embodiments, the pores do not communicate with granule surface. In some embodiments, varying sizes of said powders or granules may be used in the adhesive composition. In some embodiments, the multivalent metal salts (e.g., tetra-calcium phosphate) may react with a compound of a Formula (e.g., Formula (I), Formula (II), Formula (III), Formula (IV), Formula (V), Formula (VI), or a combination thereof) to form an adhesive composition when combined with an aqueous medium.

In some embodiments, the therapeutic may comprise drug(s) used for pain relief, infection control and prevention, chemotherapy, osteoporosis treatment, tissue regeneration, imaging, or a combination thereof. In some embodiments, the therapeutic can be premixed as a solid. The solid can be of all size ranges varying from fine powder or granules. In some embodiments, the drug can be mixed as a solution. The solution can be used independently or mixed with the aqueous medium. In some embodiments, the drugs can be in the acid form or base form, salt form or combination thereof.

In some embodiments, the aqueous medium comprises water (e.g., sterile water), saliva, buffers (e.g., sodium phosphate, potassium phosphate, or saline (e.g., phosphate buffered saline)), blood, blood-based solutions (e.g., plasma, serum, bone marrow), spinal fluid, dental pulp, cell-based solutions (e.g, solutions comprising fibroblasts, osteoblasts, platelets, odontoblasts, stem cells (e.g., mesenchymal stem cells) histiocytes, macrophages, mast cells, or plasma cells), or combinations thereof in the form of aqueous solutions, suspensions, and colloids. In some embodiments, the aqueous medium comprises sterile water, distilled water, deionized water, sea water, or fresh water.

In some embodiments, the composition is prepared initially from dry components (e.g., the multivalent metal salt, the compound of any one of Formulas (I), (II), (III), (IV), (V), or (VI), or a salt thereof, and the therapeutic. In some embodiments, said dry components composition are present in the form of a powder or granule.

### Compounds

In some embodiments, the adhesive bone regenerative compositions disclosed herein may comprise a multivalent metal salt (e.g., tetra-calcium phosphate, alpha tricalcium phosphate), a compound of a Formula (e.g., Formula (I), Formula (II), Formula (III), Formula (IV), Formula (V), or Formula (VI)) or combinations thereof, a therapeutic and an aqueous medium.

Compositions disclosed herein may comprise a compound of Formula (I):

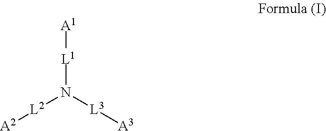

wherein: each of A¹, A², and A³ is independently selected from an acidic group (e.g., a carboxyl or phosphonyl); and each of L¹, L², and L³ is independently bond, alkylene (e.g., C₁-C₆ alkylene), or heteroalkylene (e.g., C₁-C₆ heteroalkylene). In some embodiments, the compound is a salt of Formula (I).

In some embodiments, each of A¹, A², and A³ is independently a carboxyl or phosphonyl. In some embodiments, A¹ is carboxyl, and A² and A³ are phosphonyl. In some embodiments, A¹, A² and A³ are phosphonyl.

In some embodiments, each of L¹, L², and L³ is C₁-C₃ alkylene. In some embodiments, each of L¹, L², and L³ is C₁ alkylene.

In some embodiments, the compound of Formula (I) is a compound of Formula (I-a) or (I-b):

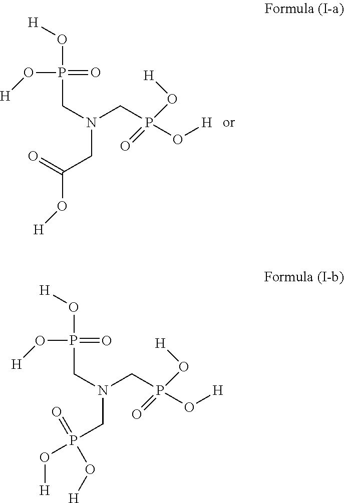 [SMILES: O=C(O)CN(CP(=O)(O)O)CP(=O)(O)O ; O=P(O)(O)CN(CP(=O)(O)O)CP(=O)(O)O]

In some embodiments, the aqueous medium is water.

In some embodiments, the composition further comprises an additive.

Compositions disclosed herein may comprise a compound of Formula (II):

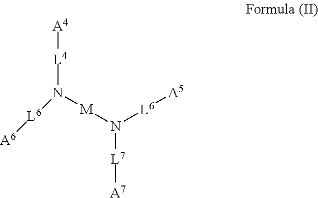

wherein: each of A⁴, A⁵, and A⁶, is independently selected from an acidic group (e.g., a carboxyl or phosphonyl); A⁷ is selected from an acidic group (e.g., a carboxyl or phosphonyl), a hydrogen atom, an alkyl, an aryl, a hydroxy group, a thio group, and an amino group; each of L⁴, L⁵, L⁶, and L⁷ is independently bond, alkylene (e.g., C₁-C₆ alkylene), or heteroalkylene (e.g., C₁-C₆ heteroalkylene); and M is alkylene (e.g., C₁-C₆ alkylene) or heteroalkylene (e.g., C₁-C₆ heteroalkylene). In some embodiments, the compound is a salt of Formula (II).

In some embodiments, A⁴, A⁵, A⁶ and A⁷ are carboxyl.

In some embodiments, L⁴, L⁵, L⁶, and L⁷ are C₁-C₃ alkylene. In some embodiments, L⁴, L⁵, L⁶, and L⁷ are C₁ alkylene.

In some embodiments, M is C₁-C₄ alkylene. In some embodiments, M is C₂ alkylene. In some embodiments, M is C₃ alkylene. In some embodiments, M is C₁-C₆ heteroalkylene. In some embodiments, M is C₆ heteroalkylene. In some embodiments, M is bis(ethyleneoxy)ethylene. In some embodiments, M includes side chains. In some embodiments, M includes multiple side chains. In some embodiments, M includes one or multiple carboxymethylene side chains. In some embodiments, M includes one or multiple N-carboxymethylene groups or N-hydroxymethylene groups.

In some embodiments, the compound of Formula (II) includes three, four, five, six, or more N-carboxymethylene groups.

In some embodiments, the compound of Formula (II) is a compound of Formula (II-a), (II-b), (II-c), (II-d), (II-e), or (II-f):

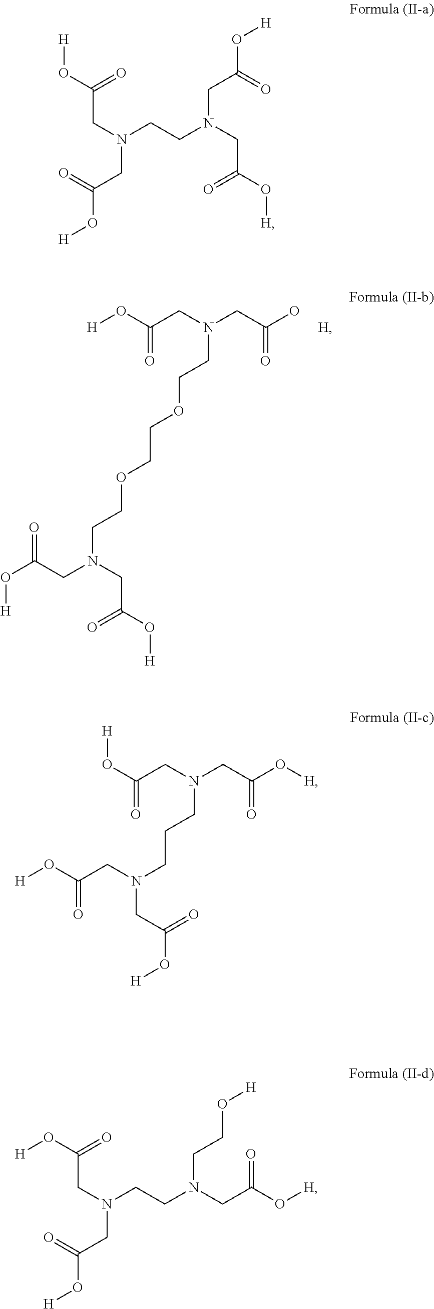

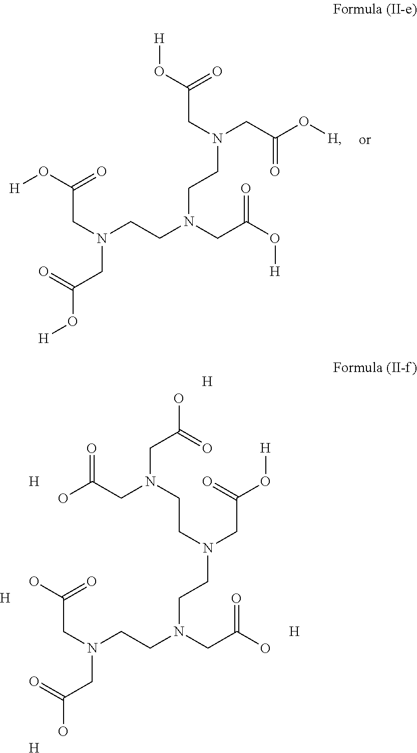

In some embodiments, the aqueous medium is water.

In some embodiments, the composition further comprises an additive.

Compositions disclosed herein may comprise a compound of Formula (III):

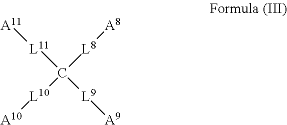

wherein: each of A⁸ and A⁹ is independently selected from an acidic group (e.g., a carboxyl or phosphonyl); each of A¹⁰ and A¹¹ is independently selected from an acidic group (e.g., a carboxyl or phosphonyl), a hydrogen atom, an alkyl, aryl, a hydroxy group, a thio group, and an amino group; each of L⁸, L⁹, L¹⁰ and L¹¹ is independently bond, alkylene (e.g., C₁-C₆ alkylene), or heteroalkylene (e.g., C₁-C₆ heteroalkylene). In some embodiments, the compound is a salt of Formula (III).

In some embodiments, A⁸, A⁹, and A¹⁰ are carboxyl.

In some embodiments, A¹⁰, A¹¹, are a hydrogen atom.

In some embodiments, A¹¹ is a hydroxy or amino group.

In some embodiments, L⁸, L⁹, L¹⁰, and L¹¹ are a bond.

In some embodiments, L⁸ and L⁹ are C₁-C₃ alkylene.

In some embodiments L¹¹ is a heteroalkylene (e.g., C₁-C₆ heteroalkylene).

In some embodiments L¹¹ is methylenethiomethylene.

In some embodiments, the compound of Formula (III) is a compound of Formula (III-a), (III-b), (III-c), or (III-d):

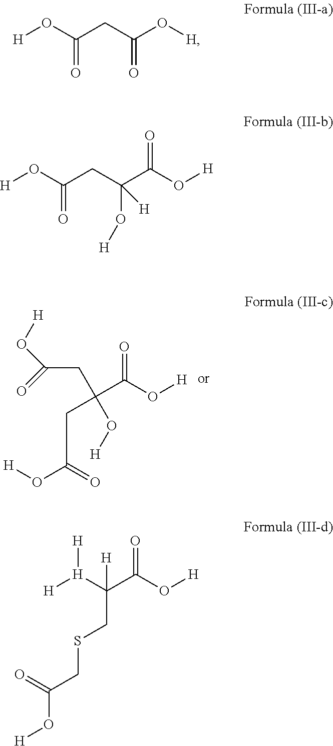

In some embodiments, the aqueous medium is water.

In some embodiments, the composition further comprises an additive.

Compositions disclosed herein may comprise a compound of Formula (IV):

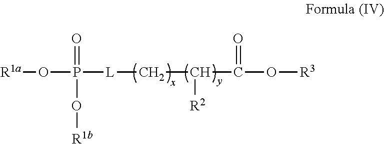

wherein: L is O, S, NH, or CH₂; each of R^{1a} and R^{1b} is independently H, an optionally substituted alkyl, or an optionally substituted aryl; R² is H, NR^{4a}R^{4b}, C(O)R⁵, or C(O)OR⁵; R³ is H, an optionally substituted alkyl, or an optionally substituted aryl; each of R^{4a} and R^{4b} is independently H, C(O)R⁶, or an optionally substituted alkyl; R⁵ is H, an optionally substituted alkyl, or an optionally substituted aryl; R⁶ is an optionally substituted alkyl or an optionally substituted aryl; and each of x and y is independently 0, 1, 2, or 3. In some embodiments, the compound is a salt of Formula (IV).

In some embodiments, L is O or S. In some embodiments, L is O. In some embodiments, each of R^{1a} and R^{1b} is independently H. In some embodiments, L is O, and each of R^{1a} and R^{1b} is H.

In some embodiments, R² is selected from H, NR^{4a}R^{4b}, and C(O)R⁵. In some embodiments, R² is NR^{4a}R^{4b}. In some embodiments, R² is NR^{4a}R^{4b} and each of R^{4a} and R^{4b} is independently H.

In some embodiments, L is O, each of R^{1a} and R^{1b} is independently H, R² is NR^{4a}R^{4b}, and each of R^{4a} and R^{4b} is independently H.

In some embodiments, R³ is H. In some embodiments, L is O, each of R^{1a} and R^{1b} is independently H, R² is NR^{4a}R^{4b}, each of R^{4a} and R^{4b} is independently H, and R³ is H.

In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1.

In some embodiments, the compound of Formula (IV) is phosphoserine.

In some embodiments, the aqueous medium is water.

In some embodiments, the composition further comprises an additive.

In another aspect, provided herein is an adhesive composition comprising a multivalent metal salt (e.g., calcium), a compound of Formula (V), a therapeutic and an aqueous medium, wherein the compound of Formula (V) is:

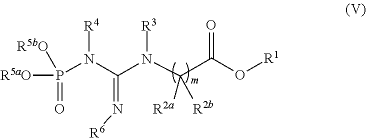

wherein R¹ is H, optionally substituted alkyl, optionally substituted alkenyl, optionally substituted alkynyl, optionally substituted aryl, or optionally substituted heteroaryl; each of R^{2a} and R^{2b} is independently H, optionally substituted alkyl, hydroxy, alkoxy, or halo; each of R³ and R⁴ is independently H or optionally substituted alkyl; each of R^{5a} and R^{5b} is independently H, optionally substituted alkyl, optionally substituted alkenyl, optionally substituted alkynyl, optionally substituted aryl, or optionally substituted heteroaryl; R⁶ is H or optionally substituted alkyl; and m is 1, 2, 3, 4, or 5. In some embodiments, the compound is a salt of Formula (V).

In some embodiments, R¹ is H. In some embodiments, each of R^{2a} and R^{2b} is independently H. In some embodiments, m is 1. In some embodiments, each of R³ and R⁴ is H. In some embodiments, each of R^{5a} and R^{5b} is independently H. In some embodiments, R⁶ is H. In some embodiments, the compound of Formula (V) is a phosphocreatine. In some embodiments, the compound of Formula (V) is Formula (V-a):

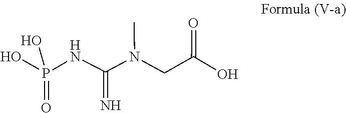

In some embodiments, the compound of Formula (V) is phosphocreatine (e.g., Formula (V-a). In some embodiments, the aqueous medium is water.

In another aspect, provided herein is a composition comprising a multivalent metal salt (e.g., calcium), a compound of Formula (VI), a therapeutic and an aqueous medium, wherein the compound of Formula (VI) is a nucleoside or nucleotide. In some embodiments, the compound of Formula (VI) is:

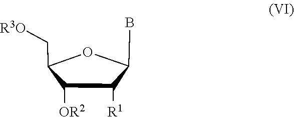

wherein B is a nucleobase; R¹ is H, OR⁴, or halo; R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl; R³ is H, optionally substituted alkyl, or a phosphate moiety (e.g., monophosphate or diphosphate); and R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl. In some embodiments, the compound is a salt of Formula (VI).

In some embodiments, B is a naturally occurring nucleobase or a non-naturally occurring nucleobase. In some embodiments, B comprises adenine, cytosine, guanosine, thymine, or uracil. In some embodiments, each of R¹, R², and R³ is H. In some embodiments, R³ is a phosphate group, e.g., a monophosphate, diphosphate, or triphosphate. In some embodiments, the compound of Formula (VI) is Formula (VI-a) or (VI-b):

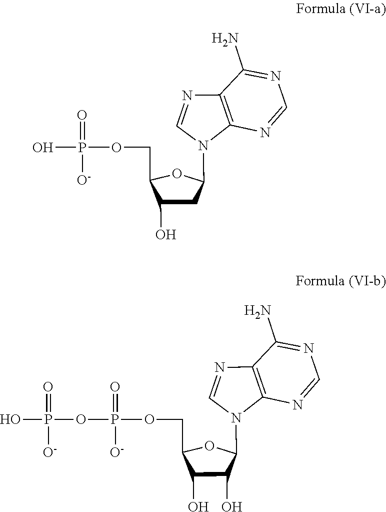

For example, the compound of Formula (VI) is 2′-deoxyadenosine monophosphate or 2′-deoxyadenosine diphosphate. In some embodiments, the aqueous medium is water.

The amount of compound of a Formula (i.e., Formula (I), Formula (II), Formula (III), Formula (IV), Formula (V), Formula (VI), or a combination thereof) may vary, e.g., between about 10% to about 90% weight by weight (w/w) of the total composition. In some embodiments, the amount of compound of a Formula (i.e., Formula (I), Formula (II), Formula (III), Formula (IV), Formula (V), Formula (VI), or a combination thereof) is in the range of about 10% to about 90%, about 15% to about 85%, about 20% to about 80%, about 30% to about 75%, about 40% to about 70%, or about 50% to about 65% w/w of the total composition. In other embodiments, the amount of compound of a Formula (i.e., Formula (I), Formula (II), Formula (III), Formula (IV), Formula (V), Formula (VI), or a combination thereof) is in the range of about 5% to about 95%, about 10% to about 85%, about 15% to about 75%, about 20% to about 65%, about 25% to about 55%, or about 35% to about 50% w/w of the total composition.

In some embodiments, the compound of a Formula (e.g., Formula (I), Formula (II), Formula (III), Formula (IV), Formula (V), Formula (VI), or a combination thereof), is initially provided as a powder or a granule. These powders may exhibit a mean particle size of about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The powder may have a mean particle size of less than about 1.000 mm. The particle size distribution may be multi-modal to include any combination of mean particle sizes as previously described. These granules may exhibit a mean granule size of about 0.050 mm to about 5 mm, about 0.100 to about 1.500 mm, about 0.125 to about 1.000 mm, about 0.125 to about 0.500 mm, about 0.125 to about 0.250 mm, about 0.250 to about 0.750 mm, about 0.250 to about 0.500 mm, about 0.500 to about 1.00 mm, about 0.500 to about 0.750 mm. The granule size may be multi-modal to include any combination of mean granule sizes as previously described. The granules may be supplied with a various proportion of porosity and a various size of internal pores. The pores may communicate with granule surface or not. In some embodiments, varying sizes of said powders or granules may be used in the adhesive composition.

The adhesive composition, including a therapeutic, is capable of undergoing sterilization, e.g., gamma irradiation, except wherein certain therapeutics may not be capable of undergoing gamma irradiation where sterilization would degrade the chemical structure of the composition. Sterilization may not compromise the chemical structure of the composition, determined by shifts in the chemical structure of the composition. Said compositions are capable of undergoing gamma irradiation up to 50 kGy, preferably between 10 and 40 kGy, or between 20 and 30 kGy.

### Therapeutics

In some embodiments, the composition comprises a therapeutic or a combination of therapeutics. For example, in some embodiments, the adhesive composition comprises a therapeutic or a combination of therapeutics. The therapeutic may be selected to treat any number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain.

In some embodiments the therapeutic is a therapeutic or a combination thereof to treat pain (e.g., pain reliever). The pain reliever may include, but is not limited to, opioids, non-steroidal anti-inflammatory (NSAIDs), local anesthetics, and gabapentinoids. Opioids may include, but are not limited to, fentanyl, morphine, hydromorphine, oxycodone, vicodin, and codeine. NSAIDs (cyclooxygenase inhibitors) may include, but are not limited to, naproxen, ibuprofen, diclofenac and acetylsalicylic acid. Local anesthetics (sodium channel blockers) may include, but are not limited to, bupivacaine, lidocaine, prilocaine, and articaine. Gabapentinoids (calcium channel blockers) may include, but are not limited to, gabapentin, pregabalin, and mirogabilin.

In some embodiments the therapeutic is a therapeutic or a combination thereof to treat infection (e.g., antibiotic). The antibiotics may include, but are not limited to, penicillins (β-lactams), cephalosporins (γ-lactams), macrolides, lincomycin, nitroimidazoles, carbapenems, fluoroquinolones, sulfonamides, tetracyclines, aminoglycosides, quinolone, polyketides, and glycopeptides. Penicillins may include, but are not limited to, amixocillin. Cephalosporins may include, but not are limited to, cefazolin, cefaclor, cephalexin and ceftriaxone. Marcolides may include, but are not limited to, erythromycin, and clarithromycin. Fluoroquinolones may include, but are not limited to, ciprofloxacin, ofloxacin. Sulfonamides may include, but are not limited to, co-trimoxazole, and trimethoprim. Tetracyclines may include, but are not limited to, tetracycline, and doxycycline. Aminoglycosides may include, but are not limited to, gentamicin, and tobramycin. Glycopeptides may include, but are not limited to, vancomycin teicoplanin, telavancin, ramoplanin and decaplanin, and bleomycin.

In some embodiments, the therapeutic is a therapeutic or a combination thereof to treat cancer (e.g., chemotherapeutic). Chemotherapeutic agents may include, but are not limited to, alkylating agents, antimetabolites, antibiotics, topoisomerase, and tyrosine kinase inhibitors. The alkylating agents may include, but are not limited to, cyclophosphamide, dacarbazine, temozolomide, and aziridine. Antimetabolites may include, but are not limited to, 5-Fluorouracil, cytarabine, gemcitabine, and methotrexate. Anticancer antibiotics may include, but are not limited to epirubicin, and idarubicin. Topoisomerase may include, but is not limited to, irinotecan, camptothecin, doxorubicin, daunorubicin, and teniposide. Tyrosine kinase inhibitor may include, but is not limited to, erlotinib, sunitinib, dasatinib, and axitinib.

In some embodiments the therapeutic is a therapeutic or a combination thereof to regenerate tissue (e.g., growth hormone, bone morphogenetic protein, hormones). Bone growth adjuvants may include citric acid. Hormones may include, but are not limited to, cholecalciferol, i.e., Vitamin D₃ form.

In some embodiments, the therapeutic is a therapeutic oiety or a combination thereof to enhance imaging. Imaging agents may include, but are not limited to, gadolinium-based contrast agent, barium-based contrast agents, and iron/iron oxide-based contrast agent. In some embodiments, an imaging agent is used in conjunction with an additional therapeutic wherein the imaging agent allows for the monitoring of the controlled released of the other therapeutic agent. For example, the contrast agent is used in conjunction with a chemotherapeutic wherein the contrast agent monitors the placement of not just the adhesive composition but also the subsequent release of the chemotherapeutic from the site of composition placement.

In some embodiments, the therapeutic can be a fine powder or granular of any size range. These powders may exhibit a mean particle size of about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. In some embodiments, the powder may have a mean particle size of less than about 1.000 mm. In some embodiments, the particle size distribution may be multi-modal to include any combination of mean particle sizes as previously described. In some embodiments, the granules may exhibit a mean granule size of about 0.050 mm to about 5 mm, about 0.100 to about 1.500 mm, about 0.125 to about 1.000 mm, 0.125 to about 0.500 mm, about 0.125 to about 0.250 mm, about 0.250 to about 0.750 mm, about 0.250 to about 0.500 mm, about 0.500 to about 1.00 mm, about 0.500 to about 0.750 mm. In some embodiments, the granule size may be multi-modal to include any combination of mean granule sizes as previously described. The granules may be supplied with a various proportion of porosity and a various size of internal pores. The pores may communicate with granule surface or not. In some embodiments, varying sizes of said powders or granules may be used in the adhesive composition.

In some embodiments, the amount of a therapeutic (e.g., pain reliever, antibiotic, anticlastic drug, growth hormone, bone morphogenetic protein, chemotherapeutic, imagine modality, or a combination thereof) in the compositions may vary, e.g., between about 0.001% to about 40% weight by weight (w/w) of the total composition. In some embodiments, the amount of a therapeutic in the compositions is in the range of about 0.1% to about 40%, about 0.001% to about 1%, about 0.1% to about 1%, about 1% to about 5%, about 5% to about 10%, about 1% to about 25%, about 2% to about 10%, about 3% to about 5%, or about 20% to about 40% w/w of the total composition.

In some embodiments, the amount of the therapeutic in the composition may be between about 0.001% to about 40% weight by weight (w/w) of the total composition. In some embodiments, the amount of a therapeutic in the compositions is in the range of about 0.1% to about 40%, about 0.001% to about 1%, about 0.1% to about 1%, about 1% to about 5%, about 5% to about 10%, about 1% to about 25%, about 2% to about 10%, about 3% to about 5%, or about 20% to about 40% w/w of the total composition.

In some embodiments, the therapeutic or a combination thereof in the adhesive composition may be released. In some embodiments, the therapeutic or a combination thereof (e.g., pain reliever, antibiotic, anticlastic drug, growth hormone, bone morphogenetic protein, chemotherapeutic, imagine modality) in the composition may be controllably released from the compositions within seconds to a minute post implantation of the composition. In some embodiments, the therapeutic may be released from the composition within weeks post implantation of the composition. In some embodiments, the therapeutic may be released from the composition within months post implantation of the composition. In some embodiments, the therapeutic may be released from the composition within years post implantation of the composition. In some embodiments, release of the therapeutic agent can be monitored via an additional imaging therapeutic released from the composition. For example, an adhesive composition comprises a combination of a chemotherapeutic, e.g., antimetabolite and an imaging therapeutic, e.g., BaSO₄, wherein the combination of therapeutics is released after delivery of the composition and can be viewed via fluoroscopy of the contrast agent.

In some embodiments, the rate of release of the therapeutic or a combination thereof from the composition, e.g., adhesive composition, may be controlled. In some embodiments, the therapeutic or a combination thereof (e.g., pain reliever, antibiotic, anticlastic drug, growth hormone, bone morphogenetic protein, chemotherapeutic, imagine modality) is released at a rate from the composition that is controlled to achieve sustained release until the composition completely biodegrades or until the therapeutic is completely released from the composition. In some embodiments, the therapeutic can have initial burst release from the composition followed by a gradual release. In some embodiments, the therapeutic can have an initial slow, gradual release from the composition followed by a burst release. In some embodiments, the rate of therapeutic release or release profile of a target therapeutic can be tailored to a specific time-dose curve using conventional time release technology such as passive diffusion. The rate of therapeutic release or release profile from the composition may be optimized based on the nature of the therapeutic (acid, base or salt). In some embodiments, the rate of therapeutic release or release rate from the composition may be controlled by altering the particle size distribution of one or more components (e.g., multivalent metal, compound of a Formula, additive) of the composition, by introducing and maximizing porosity and interconnected pores into the matrix and by minimizing the therapeutic interaction with other compounds in the composition. In some embodiments, the rate of therapeutic release or release profile of a target therapeutic can be tailored to a specific time-dose curve governed by a combination of the factors described above (e.g., interaction with adhesive composition, resorption, diffusion, particle size, porosity, pore size).

The present disclosure provides that the therapeutic may be controllably released so that the therapeutic release follows a specific time-dose curve. In some embodiments, this is achieved by forming the pores during the setting process of the composition to increase the surface area of the composition and therefore the diffusion rate. More specifically, a salt additive (e.g., calcium carbonate, calcium bicarbonate, sodium carbonate, or sodium bicarbonate) is included in the composition wherein said additive reacts in the composition to form pores and porosity during the in situ hardening or curing process of the adhesive composition. More specifically, as the additive reacts with the multivalent metal salt and compound present in the composition, carbon dioxide gas is released in the form of bubbles which form pores and cause the adhesive composition to expand while hardening. The amount of additive included in the composition directly correlates to how porous the adhesive composition is. In some embodiments, the amount of this additive in the composition may range from about 0.5% to about 20% w/w of the solid components of the adhesive composition. In some embodiments, the porosity of the adhesive composition ranges from about 5% to about 95% in direct correlation to the amount of additive present in the composition. The therapeutic present in the adhesive composition is able to move diffusely through the pores and to the defect site or throughout the body.

In some embodiments, the method of controlling the release of the therapeutic comprises including porous granules as an additive of the adhesive composition wherein said porous granules consist of a solidified form of the adhesive composition wherein said pores are created in the same method as described above. In some embodiments, said granules comprise an amount of the therapeutic and said therapeutic is released through the granule additives at the defect sites. In some embodiments, porous granules are present in addition to the adhesive composition itself being porous via the method described above, wherein the therapeutic is released from both the porous composition and the porous granule additives.

In some embodiments, the release rate of the therapeutic out of the composition can be determined by the rate of composition biodegradation, wherein the release rate of the therapeutic follows a linear model along with the biodegradation or bioabsorbtion of the adhesive composition. In other embodiments, the release rate of the therapeutic out of the composition is determined by the partition coefficient of the specific therapeutic. A partition coefficient is a measurement of the affinity of a therapeutic to the composition, i.e., the ratio of the amount of drug released from the composition to the ratio of the amount of drug retained in the composition under specific positions. The higher the partition coefficient is, the shorter the release time is. The partition coefficients of various therapeutics were calculated in Example 2.b below and can be found in Table 3.

In some embodiments, the therapeutic is present in the adhesive composition as an additive wherein: the therapeutic is mixed into a solidified form of the adhesive composition via the method described above; said solidified form is preformed during the putty state into a preferred state (e.g., disc, plug, cylinder, rod, bar, mesh, etc.); and said preformed composition is surgically implanted into or onto the defect site (e.g., bone or bone surface). Said preformed composition may be affixed in place via a compression or wedge fit, with an additional amount of non-preformed adhesive composition, using hardware (e.g., screws), or any combination thereof. In some embodiments, the preformed composition could be in a predefined geometric shape for a specific application, or made a custom size based on a patient specific anatomic site.

The adhesive composition, including a therapeutic, is capable of undergoing sterilization, e.g., gamma irradiation, except wherein certain therapeutics may not be capable of undergoing gamma irradiation where sterilization would degrade the chemical structure of the composition. Sterilization may not compromise the chemical structure of the composition, determined by shifts in the chemical structure of the composition. Said compositions are capable of undergoing gamma irradiation up to 50 kGy, preferably between 10 and 40 kGy, or between 20 and 30 kGy.

### Additives

In some embodiments, the adhesive composition may further comprise an additive. An additive may be used to impart additional functionality to the composition of the disclosure, such as improving or affecting the handling, texture, durability, strength, or resorption rate of the material, or to provide additional cosmetic or medical properties. In some embodiments, an additive may add pores and increase porosity of the composition. Exemplary additives may include salts (e.g., calcium carbonate, calcium bicarbonate, sodium carbonate, sodium bicarbonate, sodium chloride, potassium chloride, sodium citrate, calcium citrate, magnesium citrate, strontium citrate), fillers, formulation bases, viscosity modifiers (e.g., polyols (e.g., glycerol, mannitol, sorbitol, trehalose, lactose, glucose, fructose, or sucrose)), abrasives (e.g., bone fragments), coloring agents (e.g., dyes, pigments, or opacifiers), flavoring agents (e.g., sweeteners). In some embodiments, the additive includes a polymer.

In some embodiments the additive is a salt, e.g., calcium carbonate, calcium bicarbonate, sodium carbonate, sodium bicarbonate, sodium chloride, potassium chloride, sodium citrate, calcium citrate, magnesium citrate, or strontium citrate. In some embodiments the additive is calcium carbonate, calcium bicarbonate, sodium carbonate, or sodium bicarbonate, wherein said additive reacts in the composition to form pores and porosity during the in situ hardening or curing process of the adhesive composition. More specifically, as the additive reacts with the components of the composition, carbon dioxide gas is released in the form of bubbles which form pores and cause the adhesive composition to expand. In some embodiments, said pores are interconnected. In some embodiments, these salt additives have a mean particle size of about 0.000005 mm to about 1.0 mm, e.g., from about 0.000005 to about 0.15 mm, about 0.25 to about 0.75 mm, 0.25 to about 0.5 mm, 0.1 to about 0.05 mm, about 0.000015 to about 0.000025 mm, about 0.02 to about 0.06 mm, about 0.02 to about 0.04 mm, about 0.04 to about 0.1 mm, about 0.04 to about 0.06 mm, about 0.06 to about 0.15 mm, or about 0.06 to about 0.125 mm. In some embodiments, these salt additives can have a multi-mode mean particle size as defined above. In some embodiments, the amount of this additive in the composition may range from about 0.5% to about 20% w/w of the solid components of the adhesive composition. In some embodiments, the porosity of the adhesive composition ranges from about 5% to about 95% in direct correlation to the amount of additive present in the composition. In some embodiments, these salt additives are mixed with the solid components of the adhesive composition or are dissolved or suspended in the aqueous medium or a combination of both.

In some embodiments, the additive is a polymer. Suitable polymers may contain functional groups that contain electronegative atoms as the bonding sites of the polymer surfaces to the available metal ions, such as electronegative carbonyl oxygen atom(s) of the ester group or electronegative nitrogen atom(s) of the amine group as the bonding sites of the polymer surfaces to the available metal ions. These functional groups can be either in the backbone chain of the polymer or in groups pendant to the polymer chain. These polymeric based compounds may include, but are not limited to, one or more of the following; poly(L-lactide), poly(D,L-lactide), polyglycolide, poly(γ-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin and/or mixtures thereof. In addition, copolymers of the above homopolymers also can be used.

In some embodiments, the general structural nature of a polymer (e.g., a polymer used as an additive in a composition, e.g., an adhesive composition, described herein) may include a linear homo and copolymer, a cross linked polymer, a block polymer, a branched polymer, a hyper branched polymer, or a star shaped polymer. In some embodiments, the polymers can be added to the composition in the form of a solution, powder, fiber, resin, mesh, liquid crystal, hydrogel, chip, flake, granule, and the like. In some embodiments, the polymeric material can be included directly within the composition or can be an adjunct that is applied in situ as the cement is applied to the bone.

In some embodiments, the additive is a solidified form of the adhesive composition present as porous granules ranging in size from about 0.1 mm to about 5 mm, e.g., about 0.2 mm to about 1 mm, about 0.3 mm to about 2.5 mm, about 0.5 mm to about 3 mm, about 0.5 mm to about 0.75 mm, or about 0.75 mm to about 4 mm. In some embodiments, the pores of said granules range in size from about 0.000005 mm to about 1.0 mm, e.g., from about 0.000005 to about 0.15 mm, about 0.25 to about 0.75 mm, 0.25 to about 0.5 mm, 0.1 to about 0.05 mm, about 0.000015 to about 0.000025 mm, about 0.02 to about 0.06 mm, about 0.02 to about 0.04 mm, about 0.04 to about 0.1 mm, about 0.04 to about 0.06 mm, about 0.06 to about 0.15 mm, or about 0.06 to about 0.125 mm. In some embodiments, said pores are formed via inclusion of an additive, e.g., calcium carbonate or sodium carbonate, wherein the reaction of the additive in the composition forms carbon dioxide gas and releases bubbles which forms the pores. The composition is allowed to set into cement form and ground up (e.g., by a jaw crusher, co-mill, jet-mill or ball-mill) into the size range described above. In some embodiments, the ground cement granules are sieved to control the size range. In some embodiments, a therapeutic as described within is included in the solidified form of the adhesive composition.

In some embodiments, the composition comprises a plurality of additives. In some embodiments, certain additives may be provided as powders, granules, solutes or any combination thereof. These powders may exhibit a mean particle size of about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. In some embodiments, the powder may have a mean particle size of less than about 1.000 mm. In some embodiments, the particle size distribution may be multi-modal to include any combination of mean particle sizes as previously described. In some embodiments, the granules may exhibit a mean granule size of about 0.050 mm to about 5 mm, about 0.100 to about 1.500 mm, about 0.125 to 1.000 mm, 0.125 to 0.500 mm, about 0.125 to 0.250 mm, about 0.250 to 0.750 mm, about 0.250 to 0.500 mm, about 0.500 to 1.00 mm, about 0.500 to 0.750 mm. In some embodiments, granule size distribution may be multi-modal to include any combination of mean granule sizes as previously described. In some embodiments, the granules may be supplied with a various proportion of porosity and a various size of internal pores. In some embodiments, the pores may communicate with granule surface. In some embodiments, the pores do not communicate with granule surface. In some embodiments, varying sizes of said powders or granules may be used in the adhesive composition.

In some embodiments, certain additives may be provided as fibers. In some embodiments, the fibers may exhibit a mean fiber diameter of about 0.010 mm to about 2 mm, about 0.010 mm to about 0.50 mm, or about 0.025 mm to about 0.075 mm. These fibers may exhibit a mean fiber length of about 0.025 mm to about 50.0 mm, about 0.50 mm to 10 mm, or about 1.00 mm to about 3.50 mm. The fiber diameter distribution or length distribution may be multi-modal to include any combination of mean fiber diameter or length.

### Properties

In some embodiments, the adhesive bone regenerative composition, optionally comprising a therapeutic, might be applied to the surface of a structure in its fluid or semi-solid state by means of an injection delivery device or by application using an instrument such as a spatula. The viscosity of the adhesive composition when in its fluid state might be as low as about 100 cP to about 10,000 cP and in its semi-solid state from about 10,000 cP to about 250,000 cP. In some embodiments, the viscosity and cohesion properties of the composition may facilitate the ability to squeeze the material through a needle or cannula as small as 18 gauge when the viscosity is in the low range of its fluid state. In some embodiments, with viscosities in the semi-solid state, the shape and amount of material can be altered through spreading or removal techniques without substantially effecting the strength of the set material. In some embodiments, the working time of the adhesive composition is when the viscosity is between about 100 cP to about 250,000 cP.

In some embodiments, the adhesive composition, optionally comprising a therapeutic, may have a tacky state after mixing with an aqueous medium. In some embodiments, this tacky property is retained for a number of days (e.g., up to 7 days, up to 3 days, up to 1 day), up to hours (e.g., up to 12 hours, up to 4 hours, up to 1 hour), up to minutes (e.g., up to 30 minutes, up to 12 minutes, up to about 4 minutes, up to about 2 minutes, up to about 1 minute), or seconds (e.g., up to 30 seconds, up to 5 seconds, up to 2 seconds), after mixing with the aqueous medium. The duration of the tacky state may be dependent on a number of factors including relative ratio of the components, the particle sizes of the component materials, the presence of additives and the like, or the temperature of the environment. In some embodiments, during the tacky state, the adhesive composition will adhere to surfaces without the need for external clamping or other application of pressure. In some embodiments, the adhesive composition in the tacky state will adhere bone to bone and bone to other materials. In some embodiments, the adhesive composition in the tacky state may adhere materials such as stainless steel, titanium, zirconia, polyether ether ketone, steel, aluminum, copper, brass, aragonite, calcite, cement, alumina, concrete, ceramics, rock, glass, and other metals or substances. In some embodiments, during the tacky state the contacting surfaces may be held together by the adhesive composition itself, without the need for external force, until the composition sets to the final hardened cement state. In some embodiments, the tacky state can allow the materials to be positioned or repositioned without appreciable loss of cured strength.

The amount of force needed to remove two adherent pieces of material from each other during the tacky state is the tack strength. In some embodiments, the composition, optionally comprising a therapeutic, when applied to join or affix two surfaces may have a tack stress, as measured by tensile or shear loads during the tacky state, from about 10 kPa to about 250 kPa and preferably from about 50 kPa to about 150 kPa. In some embodiments, the tack stress may be sufficiently high that the items to be joined need not be held or clamped together unless there is an opposing strength of the items greater than the tack strength. During the tacky state the materials may be positioned, repositioned or reopposed several times without appreciable loss of cured adhesive strength.

In some embodiments, the adhesive composition, optionally comprising a therapeutic, may adopt a pliable working or putty state after mixing with an aqueous medium prior to hardening, which is present for up to about one week or less, one day or less, one hour or less, 30 minutes or less, depending on the components of said compositions and the conditions of the application, e.g., temperature. In some embodiments, the composition may adopt a pliable working or putty state for less than or equal to about one week after mixing with an aqueous solution or suspension, e.g., less than about six days, less than about five days, less than about four days, less than about three days, less than about two days, less than about one day, less than about twelve hours, less than about one hour, less than about 30 minutes, less than about 20 minutes, less than about 15 minutes, less than about 10 minutes, less than about 5 minutes, less than about 3 minutes, less than about 2 minutes, less than about 1 minute, less than about 30 seconds, less than about 5 seconds after mixing with an aqueous solution or suspension.

In some embodiments, during the putty state, which follows the tacky state, the adhesive composition, optionally comprising a therapeutic, can be shaped or sculpted, for example, to fill voids in bone or acquire a desired contour, size or form. The combined time of the tacky state and the putty state is referred to herein as working time. In some embodiments, the adhesive bone regenerative composition may have a working time of up to at least 3 minutes, up to at least 5 minutes, up to at least 8 minutes, up to at least 12 minutes, or up to at least 15 minutes from initial mixing, after which time the compositions have sufficiently begun hardening.

In some embodiments, after a set amount of time, the adhesive composition, optionally comprising a therapeutic, may adopt a hard, cement-like state. This process of conversion from the pliable working state to the cement-like state may be referred to as “hardening,” “curing,” or “setting.” In some embodiments, the adhesive composition may harden, cure, or set such that the materials that have been affixed to each other with the adhesive compositions cannot be separated without the application of significant force. In some embodiments, the adhesive compositions will begin to harden within about 8 minutes, e.g., within about 5 minutes, within about 3 minutes, or within about 15 minutes, after mixing with the aqueous medium near room or body temperature. In some embodiments, adhesive compositions may be formulated to harden within a specific amount of time. For example, certain formulations may harden within less than 8 minutes, e.g., less than 30 seconds. Other formulations may harden within more than 8 minutes, for example, more than about 12 minutes, more than about 15 minutes, more than one day or about one week. The variance in hardening times may be due to the composition or the environment (e.g., temperature). In some embodiments, hardening time may range between less than 30 seconds to more than one day, under the same external conditions. In some embodiments, the described tacky, putty, and set state can occur in a wet environment or dry environment.

In some embodiments, the adhesive composition optionally comprising a therapeutic may exhibit an adhesive strength in the cement-like state in the range of about 100 kPa to about 12,000 kPa, depending on the application and the particular components and ratios of components in said adhesive compositions. In some embodiments, the adhesive strength of the adhesive composition in the cement-like state is between about 100 kPa and e.g., about 10,000 kPa, about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the adhesive composition in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the adhesive composition in the cement-like state is in the range of about 200 kPa and about 2,500 kPa. In some embodiments, the adhesive strength of the adhesive composition in the cement-like state is greater than 100 kPa.

In some embodiments, the particular components of the adhesive composition may be selected to achieve the desired strength depending on the intended use of the adhesive compositions. In an embodiment, a skilled practitioner (e.g., a doctor, dentist, surgeon, nurse, medic, emergency technician, carpenter, mechanic, plumber, or other suitable person) may alter the specific components to achieve the desired adhesive properties of said composition based on the intended use or desired outcome.

In some embodiments, the adhesive compositions optionally comprising a therapeutic described herein may be applied to surfaces that are dry, immersed in, submerged in, or damp with an aqueous medium (e.g., fresh water, saline, blood, sea water), or condensing water.

### Medical Uses of the Compositions

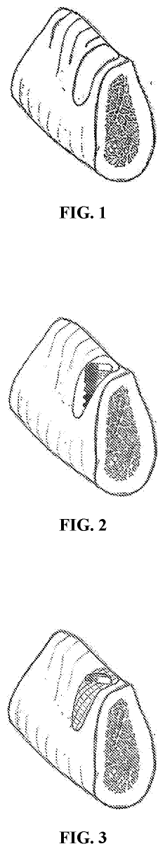

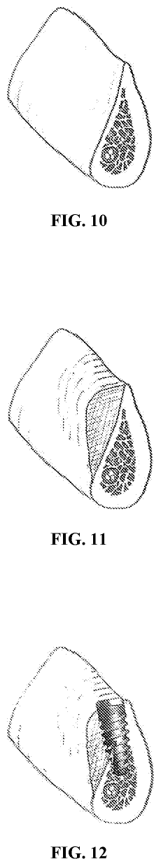

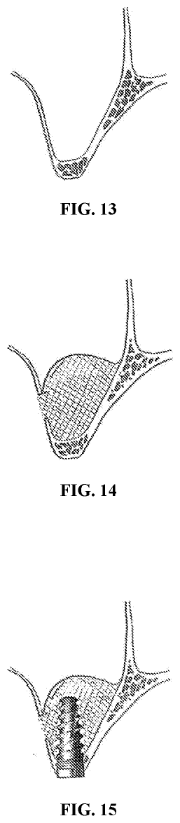

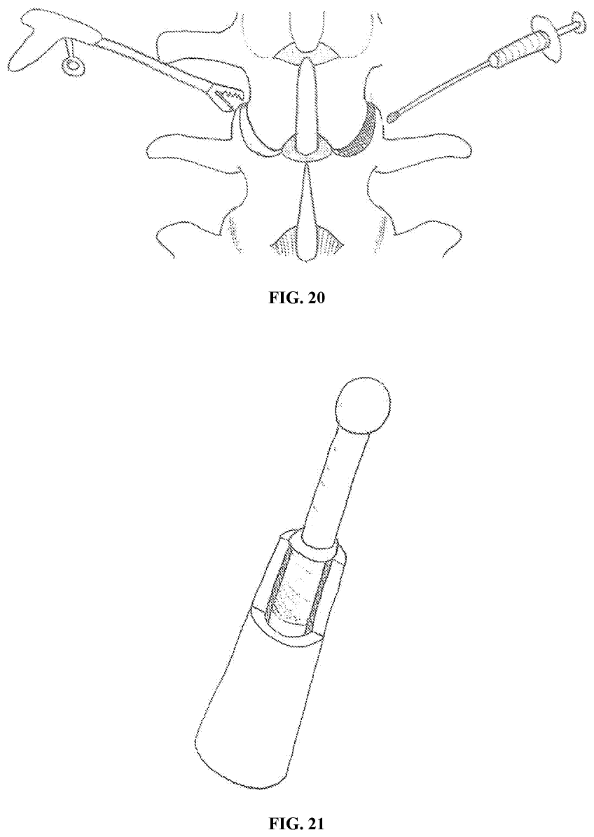

The adhesive composition optionally comprising a therapeutic may be useful in a wide variety of applications, for example, medical applications. In some embodiments, the composition may be used to adhere a structure to a surface (e.g., a bone, a tooth, or another structure, e.g., as shown in FIGS. 1-20), for which the bone regenerative composition occludes access to the bone or bone wound surface to undesirable factors or for which the therapeutic is either retained or locally and controllably released from the composition to treat any one of a number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. In some embodiments, the structure comprises an implant, anchor, graft, device, biological tissue, prosthetic device, dental crown, or another bone or bone fragment. In some embodiments, the surface is the endosseous surface or the subperiosteal surface of the bone. In some embodiments, the adhesion of said structure is temporary, such that said structure is removed after a period of time (e.g., greater than about 1 hour, about 2 hours, about 12 hours, about 24 hours, about 1 week, about 1 month, about 6 months, about 1 year, about 5 years). In other embodiments, the adhesion of said structure is permanent or intended to be permanent or until the material is resorbed and replaced with bone.

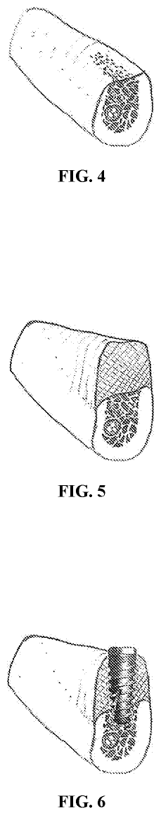

In some embodiments, the adhesive composition may be used to fill a gap, hole, or void in a surface. The compositions may be used either before (e.g., a bone or other structure, e.g., as shown in FIGS. 4-15) or after placement of a structure into or onto a surface (e.g., implant, anchor, graft, device, biological tissue, or another bone or bone fragment, e.g., as shown in FIGS. 1-3), wherein the composition occludes access to the bone or bone wound surface to undesirable factors and the therapeutic may be either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition will may mitigate post-operative pain. It is envisioned that this particular application may be useful when the core diameter of the gap, hole, or void is larger than the size of the structure, for instance, in the placement of a dental implant into the alveolar ridge (e.g., as shown in FIGS. 1-3). In this instance, the adhesive composition may impart additional strength, fixation, stability, durability, or other advantageous property to the attached structure at the attachment site. In other embodiments, the composition may impart fixative strength to the attached structure at the attachment site (e.g., as shown in FIG. 17); for which the composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. In some embodiments, the adhesive composition may support new bone growth at the attachment site, e.g., by increasing or stimulating the bone resorption, deposition, or the remodeling rate. In additional embodiments, the adhesive composition may be used to fill gaps created in or at the attachment site to create a seal (e.g., as shown in FIGS. 1-3 and FIG. 22), prevent leakage, or prevent microbial growth (e.g., an infection), or prevent fibrous tissue ingress. In some embodiments, the adhesive composition may be used to prevent microbial ingress, e.g., to prevent an infection, e.g., to prevent fibrous tissue ingress (e.g., as shown in FIGS. 1-3 and FIG. 22).

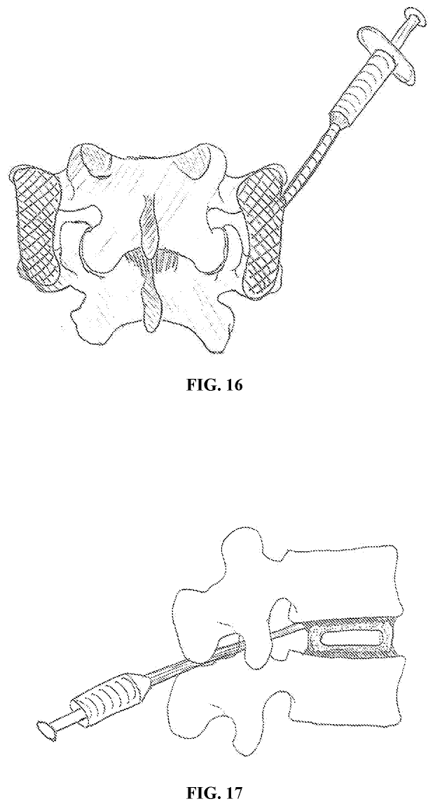

In some embodiments, the adhesive composition optionally comprising a therapeutic may be used during a medical procedure. In some embodiments, the adhesive composition may be used during a surgical or dental procedure. In some embodiments, said medical procedure comprises a surgery (e.g., dental surgery, sinus surgery, facial surgery, or other surgery to the skeletal region). In other embodiments, the medical procedure comprises a spinal fusion, prosthetic limb attachment, skeletal cosmetic augmentation, filling of a defect, a void, or a deficiency, or other surgery to the musculoskeletal system. The surgical procedures may be through open tissue procedures to expose or gain access to the application site. Likewise, the surgical procedures may be through small incisions or through minimally invasive approaches in order to minimize tissue damage (e.g., with needle access to an application site and to inject an exemplary composition during spinal fusion procedures as shown in FIGS. 16-20). Such surgical procedures could be performed in sterile operating rooms or in non-sterile settings such as at a clinic, office setting, or at a remote site outside a hospital setting (e.g., injury site, battlefield, ocean, ambulance).

In some embodiments, the adhesive composition optionally comprising a therapeutic may be used in a dental application (e.g., ridge preservation graft following tooth extraction, filling of a cavity or defect resulting from wisdom tooth removal, tooth decay, fracture, attrition, abrasion, erosion, abfraction, placement of a dental implant or device, osteoperiosteal graft, endodontic reconstruction, or others). For example, the adhesive composition may be used in a dental application. In particular embodiments, the compositions may be used in a dental application for which the composition, e.g., bone regenerative composition, occludes access to the bone or bone wound surface to undesirable factors or for which the therapeutic is either retained or locally and controllably released from the composition to treat any number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. In some embodiments, the adhesive composition may be used as an onlay graft to increase bone volume (e.g., as shown in FIGS. 4-16 and FIG. 18). In other embodiments, the composition optionally comprising a therapeutic may be used in ossicular chain reconstruction or to adhere a structure to the inner ear or middle ear, e.g., a hearing aid. In other embodiments, the adhesive composition may be used to provide contour in a surgical application, e.g., for facial bone augmentation applications, for which the composition occludes access to the bone or bone wound surface to undesirable factors or for which the therapeutic is either retained or locally and controllably released from the composition to treat any number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. In some embodiments, the adhesive composition may be used in a cosmetic application.

In other embodiments, the adhesive composition may be placed by injection into an extraction socket and allowed to become solid. In some embodiments, the solid material may be allowed to remain undisturbed until the composition hardens, cures, or resorption of the material proceeds, resulting in increased bone volume through alveolar fill graft and ridge preservation.

In some embodiments, the adhesive composition optionally comprising a therapeutic may be placed or injected into a bone void. The bone void may be one resulting from the removal of a bone cyst, or granuloma, or similar bone defect crestal, central, or lateral to the alveolar ridge (e.g., bone) or another portion of the facial skeleton, and allowed to become solid, for which the composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. The solid material may be allowed to remain undisturbed until the gradual resorption or dissolution of the material proceeds, resulting in simultaneous new bone generation and replacement of the original solid volume (e.g., volume maintenance), that may have increased bone strength and restoration of the bone contour.

In some embodiments, the adhesive composition, optionally comprising a therapeutic, may be placed or injected into a bone void partially or totally surrounding the superficial aspect of a structure (e.g., an implant) as it emerges from the bone, and allowed to become solid, thereby providing continuous contour to the surface of the bone, augments the stability of the structure (e.g., implant) in the bone if needed, and for which the bone regenerative composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. In some embodiments, the composition may be placed or injected into a bone void partially or totally surrounding the superficial aspect of a structure to seal a gap to exclude fibrous tissue or prevent microbial ingress (e.g., prevent an infection, e.g., as shown in FIGS. 1-3 and FIG. 22). In some embodiments, this might be performed during an initial visit after an implant is placed. In some embodiments, this might be performed during a rescue procedure of an implant. The solid material may be allowed to remain undisturbed until the gradual resorption or dissolution of the material proceeds, resulting in simultaneous new bone generation and replacement of the original solid volume (e.g., volume maintenance), that may have increased bone strength and restoration of the bone contour.

In some embodiments, the adhesive composition optionally comprising a therapeutic may be placed or injected into a prepared extraction socket, or a similar bone void. In some embodiments, a structure (e.g., an implant) may be placed into this preparation in desired relationship to the surroundings before the composition becomes solid and for which the composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain. Once the composition becomes solid, the structure (e.g., an implant) possesses primary stability. In some embodiments, this may mean that the implant body is clinically immobile relative the bone host site in lateral and axial load, and in torsional load of at least 10 Ncm (e.g., 15 Ncm) of clockwise or counterclockwise rotation. In some embodiments, primary stability may mean that the implant body is clinically immobile relative the bone host site when using a Ostell meter that uses Resonance Frequency Analysis with an ISQ value measured, wherein the ISQ scale is normalized from 0-100, wherein the higher the ISQ the more stable the implant, and/or wherein an ISQ value >50 has been clinically accepted to indicate the implant is sufficiently stable to allow for loading. In other embodiments, primary stability refers to the relative immobility of the adhered surfaces that persist when the adhesive bond is subjected to load-bearing stress of at least 250 kPa. The solid material may be allowed to remain undisturbed until the gradual resorption or dissolution of the material proceeds, resulting in simultaneous new bone generation and replacement of the original solid volume (e.g., volume maintenance), that may have increased bone

In some embodiments, the placement of endosseous structures may be performed through open procedures involving partial or full thickness flap reflection or performed through flapless procedures with minimal periosteal reflection (e.g., punch access, laser, electro cautery, direct drilling, etc.).

In some embodiments, the placement of the adhesive composition, optionally comprising a therapeutic in contact with bone might be performed through open procedures involving partial or full thickness flap reflection or performed through flapless procedures with minimal soft tissue incision or interruption (e.g., injection through cannula or needle following a tunneling approach to gain access).

In some embodiments, the adhesive composition optionally comprising a therapeutic might be applied to the surface of a structure in its fluid or semi-solid state by means of an injection delivery device or by application using an instrument such as a spatula. For example, the adhesive composition may be applied in its fluid or semi-solid state by delivery device or instrument. The viscosity of the composition when in its fluid state might be as low as about 100 cP to about 10,000 cP and when it reaches its semi-solid state from about 10,000 cP to about 250,000 cP. The viscosity and cohesion properties of the adhesive composition will facilitate the ability to squeeze the material through a needle or cannula as small as 18 gauge when the viscosity is in the low range of its fluid state. With viscosities in the semi-solid state, the shape and amount of material can be altered through spreading or removal techniques without substantially effecting the strength of the set material. In some embodiments, the working time of the composition is when the viscosity is between about 100 cP to about 250,000 cP.

In some embodiments, the adhesive composition, optionally comprising a therapeutic might be applied to a surface of a host structure in its fluid or semi-solid state and remain in these states during the subsequent placement of another structure in contact with the composition before the composition hardens to a solid, whereupon the structure possesses primary stability.

In some embodiments, the adhesion of structures to a host structure may be performed into a bed, mantle, or layer of the composition that surrounds, contacts, or embeds the structure while the composition is in a fluid or semi-solid state prior to hardening, whereupon the structure possesses primary stability (e.g., as shown in FIGS. 1-3, 13-15, and 21-22). To do so, the host site for the structure may first require a preparation prior to application of the adhesive composition. The composition may be applied to the host site which may have a convex or concave or a combination of concave and convex surfaces. In some embodiments, the composition may have sufficient cohesion and adhesion to the host site while in its fluid or semi-solid state to resist displacement from gravity, hydrostatic pressure, or fluid flow acting upon it. In some embodiments, the structure may be placed into or onto the composition while in its fluid or semi-solid state in a desired location relative to the surroundings before the composition becomes solid, whereupon the structure possesses primary stability. Alternatively, the structure might be placed into or onto the host site in a desired location relative to the surroundings and subsequent to this a composition might be injected around or through a cannulation or orifice feature of the implant device that which communicates to the surface of the host site before the composition hardens, whereupon the structure possesses primary stability. Alternatively, the structure might be first coated on some or all of its surfaces with a composition and subsequently placed onto or into the host site before the fluid composition becomes a solid, whereupon the structure possesses primary stability. The solid material may be allowed to remain undisturbed until the gradual resorption or dissolution of the material proceeds, resulting in simultaneous new bone generation and replacement of the original solid volume (e.g., volume maintenance), that may have increased bone.

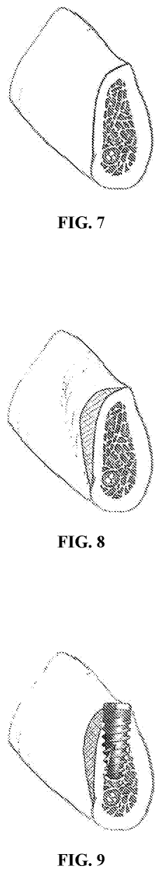

In some embodiments, the alveolar or residual ridge is augmented with compositions optionally comprising a therapeutic to create an increase in ridge bone composite volume concurrent with placement of a dental implant per the following technique. For example, the alveolar or residual ridge may be augmented with an adhesive composition. As used herein, “alveolar” and “residual” ridge refer to the bony ridge of the maxilla or mandible that contains the alveolar sockets. Generally, the alveolar ridge is referred to as the residual ridge once teeth are lost. As used herein, alveolar ridge and residual ridge may be used interchangeably to refer to the dental ridge. First, the alveolar or residual ridge may require an initial step to prepare the bone surface, which may include drilling or reaming the available bone to a desired state (e.g., as shown in FIGS. 4, 7, and 10). Next, an adhesive composition is applied to the alveolar or residual ridge (e.g., as shown in FIGS. 5, 8, and 11). A dental implant is placed into the composition in a desired location relative to the surroundings before the composition hardens to the solid state, whereupon the implant possesses primary stability. In some embodiments, the implant may partially or may not engage bone in the alveolar ridge that was previously prepared (e.g., as shown in FIGS. 6, 9, and 12). Further, the healing, temporary, or definitive abutment, and a temporary or definitive crown may be placed on the dental implant either during the same procedure at a subsequent step or at a subsequent procedure after a sufficient time interval has elapsed for soft tissue and bone healing to occur. In some embodiments, the interval might be equal to or less than twenty-four hours, twenty-four hours to one week, one week to two weeks, two weeks to one month, one month to three months, three months to six months, six months to one year, or more than one year. The crown, implant, abutment, material and surrounding bone are all subjected and able to maintain primary stability when subjected to patient loading (e.g., chewing forces).

In some embodiments, the adhesion of structures might be performed into the hardened composition following an interval. The interval might be equal to or less than an hour, more than an hour but less than eight, eight hours to twenty-four, twenty-four hours to one week, one week to two weeks, two weeks to one month, one month to three months, three months to six months, six months to one year, or more than one year. To do so, a preparation may be milled (e.g., drilled) into the substrate composed of the solidified adhesive composition and the underlying or surrounding bone. In some embodiments, the structure might be placed directly into the preparation in a desired location relative to the surroundings, whereupon the structure possesses primary stability (e.g., as shown in FIGS. 3, 6, 9, 12, and 15). In some embodiments, the preparation might be first partially or totally filled with a composition and the structure might be subsequently placed into the preparation in a desired location relative to the surroundings while the composition is in its fluid or semi-solid state and whereby the structure displaces the composition during placement before the fluid adhesive composition hardens, whereupon the structure possesses primary stability (e.g., as shown in FIGS. 3, 6, 9, 12, and 15). In some embodiments, the structure may be placed into the preparation in a desired location relative to the surroundings and subsequent to this a composition might be injected through a cannulation or orifice feature of the implant device that which communicates to the surface of the preparation before the composition hardens, whereupon the structure possesses primary stability. In some embodiments, the structure may be first coated on some or all of its surfaces with a composition and subsequently placed into the preparation before the fluid composition hardens, whereupon the structure possesses primary stability.

In other embodiments, the alveolar or residual ridge is augmented with a composition to create an increase in ridge volume (width or height) concurrent with placement of a dental implant per the following technique. For example, the alveolar or residual ridge may be augmented with an adhesive composition. In some embodiments, the alveolar ridge may require an initial step to prepare the bone surface, which may include drilling or reaming the available bone to a desired state (e.g., as shown in FIGS. 4, 7, and 10). In some embodiments, the compositions may then be placed or injected into a prepared extraction socket and is allowed to become solid. A preparation can then be milled (e.g., drilled) into the substrate composed of the solidified material and the surrounding bone. Then adhesive compositions of either similar or different compositions in their fluid state (i.e., working state) are placed into the preparation and/or applied onto the surface of the implant (e.g., dental implant). The implant is placed into the preparation in a desired location relative to the surroundings before the fluid material hardens to the solidified cement state, whereupon the implant possesses primary stability (e.g., as shown in FIGS. 6, 9, and 12). Further, the healing, temporary, or definitive abutment, and perhaps a temporary or definitive crown may be placed on the dental implant either during the same procedure at a subsequent time interval or at a subsequent procedure after sufficient time has elapsed for soft tissue and bone healing to occur to a desired state. The interval might be equal to or less than twenty-four hours, twenty-four hours to one week, one week to two weeks, two weeks to one month, one month to three months, three months to six months, six months to one year, or more than one year. In some embodiments, the crown, implant, abutment, material and surrounding bone are all subjected and able to maintain primary fixation when subjected to immediate patient loading (e.g., chewing forces). In other embodiments, the crown, implant, abutment, material and surrounding bone are all subjected and able to maintain primary stability when subjected to patient loading (e.g., chewing forces).

In some embodiments, a full thickness incision (e.g., a distant full thickness incision) followed by a tunneling subperiosteal dissection and a subperiosteal placement or injection of the adhesive composition in contact with bone are used to produce augmentation of the bone volume in the area. This might be a widening of the alveolar ridge where a dental implant placement is desired, but where the width of the residual ridge of bone is marginally insufficient for implant placement. The ridge so augmented is sufficiently broad for an osteotomy to be performed within the original bone volume, either at the time of the original procedure or after a delay of days, weeks, or months, and for the adhesive composition placed, in the hardened state or as altered by the host, to provide resistance to lateral movement of the rotary cutting instrument during bone preparation and to the implant on placement into its planned relationship to the surrounding host bed.

In some embodiments, with an alveolar residual ridge deficient in width thus limiting implant placement options, a full thickness incision and flap reflection are made, followed by application of an adhesive composition in contact with bone, are used to produce augmentation of the bone volume in the area (e.g., as shown in FIG. 11). This might be a widening of the alveolar ridge where a dental implant placement is desired, but where the width of the residual ridge of bone is initially marginally insufficient for implant placement. The ridge so augmented is as a result then sufficiently broad for an osteotomy to be performed within the original bone volume, either at the time of the original procedure or after a delay of days, weeks, or months, and for the adhesive composition placed, in the hardened state or as altered by the host, to provide resistance to lateral movement of the rotary cutting instrument during bone preparation and to the implant on placement into its planned relationship to the surrounding host bed.

In other embodiments, a full thickness incision (e.g., a distant full thickness incision) followed by a tunneling subperiosteal dissection and a subperiosteal application of an adhesive compositions optionally comprising a therapeutic in contact with bone are used to produce augmentation of the bone volume in the area (e.g., as shown in FIGS. 8 and 11), for which the bone regenerative composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. This might be increasing the height of the alveolar ridge where a dental implant placement is desired, but where the height of the residual ridge of bone is initially marginally insufficient for implant placement (e.g., as shown in FIG. 5). The augmented ridge composed of the original bone and the composition is as a result then sufficiently high for an osteotomy to be performed, either immediately or after a delay of hours, days, weeks, or months, and for the composition placed, in the hardened state or as altered by the host, to provide resistance to lateral movement of the rotary cutting instrument during bone preparation and to the implant on placement into its planned relationship to the surrounding host bed.

In some embodiments, with an alveolar residual ridge deficient in height thus limiting implant placement options, a full thickness incision and flap reflection are made, followed by application of an adhesive composition in contact with bone, are used to produce augmentation of the bone volume in the area (e.g., as shown in FIG. 5). This might be to increase the total height of available volume for dental implant placement. The volume so augmented is as a result then sufficiently tall for an implant recipient site to be milled into it, either immediately or after a delay of hours, days, weeks, or months, and for an adhesive composition placed, in the hardened state or as altered by the host, to provide resistance to lateral movement of the rotary cutting instrument during bone preparation and to the implant on placement into its planned relationship to the surrounding host bed.

In some embodiments, the ridge so augmented is sufficiently broad for an osteotomy to be performed within the volume of the wider solid composed of the bony ridge and the additional adherent material, in the hardened state or as altered by the host. In some embodiments, the implant host bed is a combination of the preexisting bone, the compositions, or the compositions as altered by the host, throughout the length of the implant. In other embodiments, the ridge so augmented is sufficiently high for an osteotomy to be performed within the volume of the higher solid composed of the bony ridge and the additional adherent material, in the hardened state or as altered by the host. Alternatively, the implant placement is delayed until the composition is partially or totally resorbed and replaced by bone. In this embodiment, the most superficial layer of the implant host site bed is the composition or the composition as altered by the host, and the deepest part of the bed is the bone volume present before the composition was placed.

In some embodiments, a subperiosteal injection or placement of the compositions optionally comprising a therapeutic in contact with bone is performed between the mucoperiosteum of a facial sinus or another air space (e.g., the nasal airway, e.g., as shown in FIG. 14) for which the adhesive composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. For example, a subperiosteal injection or placement of adhesive composition may be performed. In some embodiments, the facial sinus comprises the paranasal sinus, e.g., the maxillary sinus. This might be an augmentation in height of the bone volume where a dental implant placement is desired, but where the height of the residual bone between the oral cavity and the sinus air space is insufficient for implant placement. The bone so augmented is sufficiently high for an osteotomy to be performed within the volume of the higher solid comprised of the bone and the additional adherent material, in the hardened state or as altered by the host. In some embodiments, the implant placement is delayed until the composition is resorbed and replaced by bone, in part or totally. In this embodiment, the deepest layer of the implant host site bed is the composition or the composition as altered by the host, and the most superficial part of the bed is the bone volume present before the material was placed.

In some embodiments, the adhesive composition optionally comprising a therapeutic might be applied to bony walls of a gap or a discontinuity between bone surfaces which are indicated for closure because the gap or the discontinuity of bone causes a disability, a dysfunction, or is otherwise undesirable. For example, adhesive compositions may be adhesively applied to bony walls of a gap or discontinuity between bone surfaces. In some embodiments, the gap or the discontinuity of bone is undesirable because of loss of load-bearing function. These may include a gap or discontinuity which is congenital (e.g., cleft palate), a result of trauma (e.g., bone fracture), result of inappropriate healing (e.g., fibrous union), a result of a resection of bone (e.g., neoplasm, necrosis, or infection), or a result of procedure involving cutting or segmentation of bone in order to change its size, shape, or contour (e.g., orthognathic procedure, or correction of deformed long bones resulting from congenital, metabolic or dietary problems). The composition might be used in conjunction with fixation devices, such as microplates, bone pins and screws, or with shape and volume preserving devices, such as titanium meshes or cages, which relate the bone fragments across the gap or exclude other tissues, or it may be used alone or in combination of several formulations of the composition including those that release substances intended to affect surrounding tissues and environment. In this embodiment, the composition performs a bridging function with respect to existing elements of the skeleton, providing continuity of contour, a mechanical connection, and preventing other tissues from proliferating while bone tissue replaces the solidified material. In some embodiments, the mechanical connection is a load-bearing connection.

In some embodiments, the disclosure features a method of reinforcing a bone (e.g., osteoporotic, osteopetrotic, or affected by osteogenesis imperfecta) at risk of fracture, the method comprising: a) preparation of an adhesive composition comprising a multivalent metal salt, a small organic phosphate compound, and optionally a therapeutic in an aqueous solution or suspension; b) application of the composition to the desired region of the bone; c) applying the device of b) attached to the bone to the desired region of bone; and d) allowing the composition to remain undisturbed until the composition is hardened or cured, during which time the adhesive composition occludes access to the bone or bone wound surface to undesirable factors or during which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof, and during which the composition is resorbed and replaced by bone. In some embodiments, the method further consists of the step of applying the adhesive composition to one or a plurality of rigid or semi-rigid devices (e.g., plates, rods, strips, fibers, or bands) comprised of metal or other biocompatible material. In some embodiments, the bone is osteoporotic, osteopenic, osteopetrotic, or affected by osteogenesis imperfecta.

In some embodiments, the disclosure features a method of repairing a fractured bone (e.g., osteoporotic femur) fracture, the method comprising: a) preparation of an adhesive composition comprising a multivalent metal salt, a small organic phosphate compound, and optionally a therapeutic in an aqueous solution or suspension; b) application of the adhesive composition to the desired region of the bone; c) applying the device of b) attached to the desired region of bone; and d) allowing the composition to remain undisturbed until the composition is hardened or cured, during which time the bone regenerative composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof, and during which time the composition is resorbed and replaced by bone. In some embodiments, the method further consists of the step of application of the adhesive composition to one, or a plurality, or rigid or semi-rigid devices (e.g., plates, rods, strips, fibers, or bands) comprised of metal or other biocompatible material. In some embodiments, the therapeutic used in the composition mitigates post-operative pain.

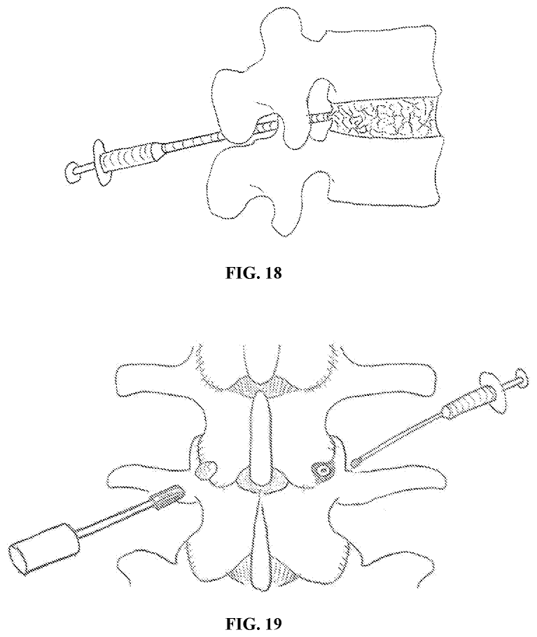

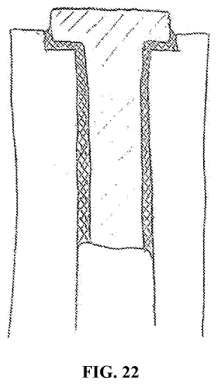

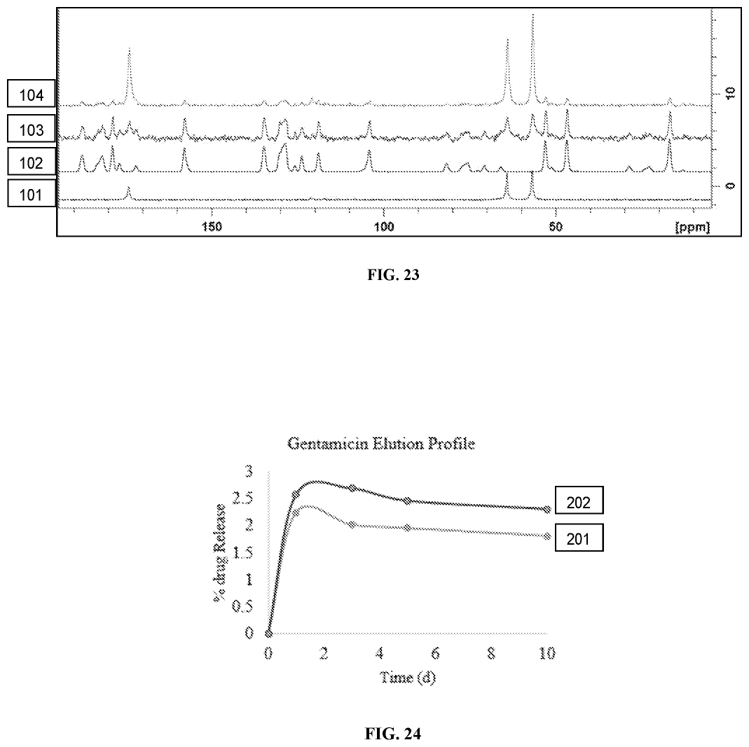

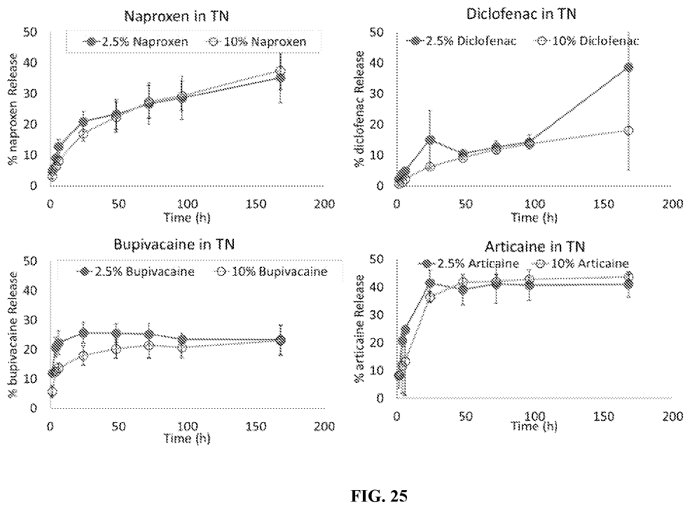

In some embodiments, the adhesive composition optionally comprising a therapeutic is applied to a gap, discontinuity, or loss of volume in bone (e.g., femur, e.g., as shown in FIGS. 21-22; alveolar ridge, e.g., as shown in FIGS. 4, 7, 10, and 13) for which the composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. For example, the adhesive composition may be adhesively applied to a gap, discontinuity, or loss of volume in a bone. In some embodiments, the therapeutic used in the composition mitigates post-operative pain. In some embodiments, the adhesive composition optionally comprising a therapeutic is applied to a gap, discontinuity, or loss of volume between bones (e.g., spinal processes, vertebral bodies as shown in FIGS. 17 and 18) for which the adhesive composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain. In some embodiments, the gap or the discontinuity causes a disability, a dysfunction, or is otherwise undesirable. The gap or discontinuity may be congenital (e.g., cleft palate), a result of trauma (e.g., bone fracture), a result of a resection of bone (e.g., neoplasm, necrosis, or infection), or a result of procedure involving cutting or segmentation of bone in order to change its size, shape, or contour (e.g., orthognathic procedure, correction of deformed long bones resulting from congenital, metabolic or dietary problems). In other embodiments, the gap or discontinuity may be a result of a resection of soft tissue (e.g., removal of cartilage or discectomy, e.g., as shown in FIGS. 17 and 18). In some embodiments, the adhesive composition is used alone. In other embodiments, the adhesive composition is used in conjunction with rigid devices, such as microplates, plates and objects of other shapes, composed of metal or other solid material, shape and volume preserving devices, such as titanium meshes, which relate the bone fragments across the gap or exclude other tissues, or it may be used alone or in combination of several formulations of the composition including those that release substances intended to affect surrounding tissues and environment. In some embodiments, the composition may be used in conjunction with interbody devices (e.g., cages, e.g., as shown in FIG. 17). For example, the composition performs a bridging function with respect to existing elements of the skeleton, providing continuity of contour, a mechanical connection, and preventing other tissues from proliferating while bone tissue replaces the solidified material.

In some embodiments, the composition optionally comprising a therapeutic is applied between articulating bone surfaces (e.g., facet joints, e.g., as shown in FIG. 19) for which the adhesive composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. For example, the adhesive composition may be applied between articulating bone surfaces. In some embodiments, the therapeutic used in the composition mitigates post-operative pain. In some embodiments, the adhesive composition optionally comprising a therapeutic is applied to adjacent bone surfaces to bridge a gap space (e.g., between vertebral bodies. e.g., as shown in FIG. 18; between transverse processes, e.g., as shown in FIG. 16; or between spinous processes), to prevent relative displacement of the bone surfaces (e.g. spinal fusion procedures), to provide load bearing support with or without supportive implants devices (e.g., screws, plates, interbody cages, e.g., as shown in FIG. 17), to occlude access to the bone or bone wound surface to undesirable factors or for which the therapeutic is either retained or locally and controllably released from the composition to treat any one of the following ailments or conditions (e.g., pain, infection, cancer, osteoporosis), to help accelerate local tissue regeneration (e.g., growth hormone, bone morphogenic protein), to assist with surgical or therapeutic treatment (e.g., imaging modality), or any of a combination thereof. In some embodiments, the therapeutic used in the composition mitigates post-operative pain. These applications might be made singly or to provide multipoint fixation when applied in several of the above or other, loci.

In some embodiments, the adhesive composition optionally comprising a therapeutic is placed or injected in contact with bone, into a space adjacent to or a gap between volumes of bone, where it is desired to affix implants or other devices (e.g., as shown in FIGS. 21-22), to occlude access to the bone or bone wound surface to undesirable factors, for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. For example, the adhesive composition may be adhesively placed or injected in contact with bone. In some embodiments, the therapeutic used in the composition mitigates post-operative pain. In some embodiments, a rigid or semi-rigid device (e.g., plate or rod) is used in conjunction with the composition, wherein the device may be placed, embedded, or otherwise affixed to the composition and the host bed at a time prior to the composition adopting the cement state, during the setting process, after the composition is solid, or after the composition is partially or totally replaced with bone tissue.

In some embodiments, the composition optionally comprising a therapeutic is placed or injected into a bone defect associated with a root of a tooth (e.g., a periodontal defect, a periapical defect), allowed to solidify; wherein the composition occludes access to the bone or bone wound surface to undesirable factors; or wherein the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality); or any of a combination thereof. For example, the adhesive composition may be placed or injected into a bone defect associated with a root of a tooth. In some embodiments, the therapeutic used in the composition mitigates post-operative pain. In some embodiments, the bone defect associated with a root of a tooth is a periodontal or periapical defect. For example, the composition. may then be replaced by bone, thus providing increased mechanical stability to the tooth and also barring environmental microbiota from access to the root surface and the interior of the alveolar socket.

In other embodiments, the composition optionally comprising a therapeutic is placed or injected into a bone defect associated with a dental implant (e.g., a periimplantitis defect) and allowed to solidify; wherein the bone regenerative composition occludes access to the bone or bone wound surface to undesirable factors; or wherein the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. For example, the adhesive composition may be placed or injected into a bone defect associated with a dental implant. In some embodiments, the therapeutic used in the composition mitigates post-operative pain. In some embodiments, the composition is resorbed and replaced by bone, thus providing increased mechanical stability to the implant and also barring environmental microbiota from accessing the implant surface and the surrounding bone.

In some embodiments, the composition optionally comprising a therapeutic has adhesive properties toward soft tissues, in addition to bone and biocompatible devices. In some embodiments, the therapeutic used in the composition mitigates post-operative pain. In some embodiments, a layer of the adhesive composition might be applied to immobilize soft tissue flaps, fragments, or zones. In some embodiments, the attachment of the adhesive composition to the soft tissues is durable enough and strong enough to close wounds. In some embodiments, the attachment of the composition provides a barrier to flow of fluids from one side of the attachment to another side. In some embodiments, the attachment of the composition provides a barrier to movement of microbes from one side of the attachment to another side. In some embodiments, the surface of the composition is a barrier to the movement of soft tissue cells (e.g., fibroblasts) into the interior of the set material. In some embodiments, the surface of the composition is a barrier to the movements of microbes into the interior of the set composition.

In some embodiments, the adhesive composition optionally comprising a therapeutic is injected or otherwise placed at the percutaneous or permucosal site of an implant or device placement to seal the site from incursion of fluids, materials, and microbiota or their products deeper into the wound, or for which the composition occludes access to the bone or bone wound surface to undesirable factors, or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. For example, the adhesive composition is injected or otherwise placed at the percutaneous or permucosal site of an implant or device placement. In some embodiments, the therapeutic used in the composition mitigates post-operative pain. This application might be contemporaneous with the initial placement of the implant or another later procedure involving the implant. The implant might be a dental implant, a maxillofacial prosthesis fixation implant or any other implant with a permucosal or percutaneous component. In some embodiments, the implant may be a prosthetic limb element.

In other embodiments, the adhesive composition optionally comprising a therapeutic might be injected, layered, sprayed, brushed, or otherwise applied to a surgical wound in areas where the bony tissue is present near the gingiva, mucosa, skin, or other element of the integument, thereby fixating the soft tissue margins and blocking movement of liquids, materials, and microbiota and their products deeper into the wound, or for which the bone regenerative composition occludes access to the bone or bone wound surface to undesirable factors, or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain.

In other embodiments, the composition optionally comprising a therapeutic may be used to reconstruct and adjoin a fissure or gap that has resulted from a congenital deformity such as, but not limited to, a cleft lip and palate. In some embodiments, the composition occludes access to the bone or bone wound surface to undesirable factors and the therapeutic may be either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain. For example, the composition could be utilized to restore the bone deformity to a primary palate by using the compositions to fill and adhere the maxillary and medial nasal processes.

In other embodiments, the adhesive composition optionally comprising a therapeutic may be used in the field of plastic surgery as an onlay graft, which can be applied to the outer surfaces of bone in the facial region. In some embodiments, the composition occludes access to the bone or bone wound surface to undesirable factors and the therapeutic may be either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain. In some embodiments, the composition may be adhesively applied as a fluid or putty like substance and contoured or molded to a desired cosmetic profile or contour before it hardens. In some embodiments, the composition may be resorbed and replaced by bone over time, while maintaining the original volume and shape formed during application. The composition could be applied, but not limited, to the chin, cheek, mid-face, or forehead regions.

In some embodiments, the composition optionally comprising a therapeutic may be adhesively applied to affix the bone fragment removed to create a window to allow access for grafting in a sinus lift procedure or a Caldwell-Luc procedure. In some embodiments, the composition occludes access to the bone or bone wound surface to undesirable factors and the therapeutic may be either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain. The adhesive composition may be injected, sprayed, brushed or otherwise applied in one or more compositional variants to fill the gap and/or affix the bony fragment or flap created by surgical instruments to allow access to the sinus cavity.

In some embodiments, the composition optionally comprising a therapeutic may be adhesively applied to affix the bone fragment or fragments removed to create access for procedures within spaces enclosed by bone (e.g., intracranial space for brain surgery procedures). In some embodiments, the composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain. In some embodiments, creating access within spaces enclosed by bone comprises providing cranial flap access to the intracranial space, e.g., for brain surgery procedures. The adhesive composition may be injected, sprayed, brushed or otherwise applied, in one or more compositional variants, to affix the bony fragment, bone fragments, or bone flap to the anatomical site from which it was removed in the course of gaining the said access by surgical instruments.

In some embodiments, the composition optionally comprising a therapeutic may be applied to obturate an opening in bone or a communication between spaces or potential spaces separated by the said bone or to occlude access to the bone or bone wound surface to undesirable factors and the therapeutic may be either retained or locally and controllably released from the composition. For example, this opening might be a congenitally, pathologically, traumatically or surgically generated bony fenestration, dehiscence, or communication (e.g., oral-antral fistula, Caldwell-Luc procedure access opening, sinus elevation graft access opening) or any other. The adhesive composition may be injected, sprayed, brushed or otherwise applied in one or more compositional variants, in conjunction or without other materials possibly serving as carrier or matrix, to occlude the passage from one to another side of the bone through gap, fistula, or communication channel.

In some embodiments, the composition optionally comprising a therapeutic is utilized as a seal to close off the communication between an intracranial or spinal space bathed in the cerebrospinal fluid and the exterior of the body for which the bone regenerative composition occludes access to the bone or bone wound surface to undesirable factors or for which the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the method of use of the composition in the previous embodiment comprises: preparation of an adhesive composition comprising a multivalent metal salt and a compound of a Formula (e.g., Formula (I), Formula (II), Formula (III), Formula (IV), Formula (V), Formula (VI), or combinations thereof), optionally a therapeutic, in an aqueous medium; application of the adhesive composition into or onto said communication, e.g., kerf, crevice, fistula, or tear; and allowing the composition to remain undisturbed until the composition is hardened, cured, or resorbed and replaced by bone. For example, the adhesive composition may serve as a seal to close off the communication between an intracranial or spinal space.

In some embodiments, the disclosure features a method of adhesively repairing a defect in a tooth, the method comprising: a) preparation of an adhesive composition comprising a multivalent metal salt and a compound of a Formula (e.g., Formula (I), Formula (II), Formula (III), Formula (IV), Formula (V), Formula (VI), or combinations thereof), optionally a therapeutic, in an aqueous medium; application of the composition into or onto said tooth defect; and allowing the composition to remain undisturbed until the composition is hardened, cured, or resorbed and replaced by bone.

In some embodiments, the adhesive composition, optionally comprising a therapeutic may be applied to the surface of a tooth for which the adhesive composition occludes access to the tooth or tooth surface to undesirable factors or for which the therapeutic is either retained or locally and controllably released from the composition. The adhesive composition, may be injected, sprayed, brushed or otherwise applied in one or more compositional variants to fill the gap in tooth substance resulting from the removal of dental caries or to a surface exposed by tooth fracture or abrasion, attrition, abfraction, or erosion of the tooth substance.

In some embodiments, the adhesive compositions, optionally comprising a therapeutic may be applied to the surface of a tooth or dental restorative material to lute or adhesively fixate them. The adhesive composition, may be injected, sprayed, brushed or otherwise applied in one or more compositional variants to fill the gap in tooth substance resulting from the removal of dental caries or to a surface exposed by tooth fracture or abrasion, attrition, abfraction, or erosion of the tooth substance.

In other embodiments, the adhesive composition optionally comprising a therapeutic are used to treat or heal a subject suffering from a disease or condition, such as cancer (e.g., osteosarcoma), osteoporosis, rickets, osteogenesis imperfecta, fibrous dysplasia, Paget's disease of the bone, hearing loss, renal osteodystrophy, a malignancy of the bone, infection of the bone, severe and handicapping malocclusion, osteonecrosis, or other genetic or developmental disease for which the bone regenerative composition occludes access to the bone or bone wound surface to undesirable factors or for which the therapeutic is either retained or locally and controllably released from the composition.

In some embodiments, the adhesive composition optionally comprising a therapeutic is used to repair a defect in a bone caused by a disease or condition, such as cancer (e.g., osteosarcoma), osteoporosis, rickets, osteogenesis imperfecta, fibrous dysplasia, Paget's disease of the bone, hearing loss, renal osteodystrophy, a malignancy of the bone, infection of the bone, or other genetic or developmental disease for which the bone regenerative composition occludes access to the bone or bone wound surface to undesirable factors or for which the therapeutic is either retained or locally and controllably released from the composition. In some embodiments, the adhesive composition optionally comprising a therapeutic used to strengthen a bone in a subject that has been weakened by a disease or condition, such as cancer (e.g., osteosarcoma), osteoporosis, rickets, osteogenesis imperfecta, fibrous dysplasia, Paget's disease of the bone, hearing loss, renal osteodystrophy, a malignancy of the bone, infection of the bone, or other genetic or developmental disease for which the bone regenerative composition occludes access to the bone or bone wound surface to undesirable factors or for which the therapeutic is either retained or locally and controllably released from the composition. In some embodiments, the subject has experienced a trauma, such as a broken bone, fractured bone, or damaged tooth. In some embodiments, the subject has experienced tooth decay. In some embodiments, the subject is undergoing a plastic surgery procedure. The compositions and methods may be used to treat a subject suffering from or afflicted with any disease or condition that impacts the structural integrity of the bony skeleton. In some embodiments, the subject is a child. In some embodiments, the subject is an adult. In some embodiments, the subject is a non-human animal.

### Additional Uses of the Adhesive Compositions

The adhesive composition described herein may be useful in a wide variety of applications, for example, non-medical applications, including industrial applications. In some embodiments, the adhesive composition may be used to adhere a structure to a surface. In some embodiments, the adhesion of said structure is temporary, such that said structure is removed after a period of time (e.g., greater than about 1 hour, about 2 hours, about 12 hours, about 24 hours, about 1 week, about 1 month, about 6 months, about 1 year, about 5 years). In other embodiments, the adhesion of said structure is permanent or intended to be permanent until the material is resorbed and replaced with another material.

In other embodiments, the adhesive composition optionally comprising a therapeutic may be used to fill a gap, hole, or void in a surface either before or after placement of a structure into or onto a surface. It is envisioned that this particular application may be useful when the core diameter of the gap, hole, or void is larger than the size of the structure. In this instance, the adhesive composition may impart additional strength, fixation, stability, durability, or other advantageous property to the attached structure at the attachment site, wherein the adhesive composition occludes access to the object or object surface to undesirable factors or for which the therapeutic is either retained or locally and controllably released from the composition. In other embodiments, the adhesive composition may impart fixative strength to the attached structure at the attachment site. In some embodiments, the adhesive composition may support new material at the attachment site. In some embodiments, the adhesive composition optionally comprising a therapeutic may be used to fill gaps created in or at the attachment site to create a seal, prevent leakage, or prevent microbial growth.

In some embodiments, the adhesive composition optionally comprising a therapeutic disclosed herein may be applied: to seal or repair a crack, fissure, leak, or defect in an object; reinforce the strength of a damaged structure; join separated objects; fill space to connect and immobilize structures (e.g., screws, trusses); or seal or secure objects that would otherwise separate due to vibration, cyclic straining, or buoyancy forces (e.g. wave action, wake, wind or current flutter, floods), rain, hail, snow, sleet, or wind; wherein the adhesive composition occludes access to the object or object surface to undesirable factors and the therapeutic may be either retained or locally and controllably released from the composition.

In other embodiments, the adhesive composition optionally comprising a therapeutic may be applied along a surface-spanning defect in an object and may be used as a patch, wherein the adhesive composition occludes access to the object or object surface to undesirable factors or wherein the therapeutic is either retained or locally and controllably released from the composition. In one embodiment, the adhesive composition optionally comprising a therapeutic may be applied along a surface-spanning crack between two objects in an object and may be used, for example, as a tape wherein the adhesive composition occludes access to the object or object surface to undesirable factors or wherein the therapeutic is either retained or locally and controllably released from the composition. In one embodiment, the adhesive composition optionally comprising a therapeutic may be applied as an interposition device between two objects and may be used, for example, as a patch or plug, wherein the adhesive composition occludes access to the object or object surface to undesirable factors or wherein the therapeutic is either retained or locally and controllably released from the composition. In some embodiments, the adhesive composition optionally comprising a therapeutic may be applied to the exterior or interior of the surface of a defect in or on an object and may be used, for example, as a patch or plug, wherein the adhesive composition occludes access to the object or object surface to undesirable factors or wherein the therapeutic is either retained or locally and controllably released from the composition.

In some embodiments, the surface to which the adhesive composition is applied comprises a metal, e.g., silicon, aluminum, titanium, cobalt, chromium, tantalum, molybdenum, copper, silver, gold, zinc, or iron. In some embodiments, the surface to which the adhesive device is applied may be a metallic coating (e.g., nickel plated, chrome placed, galvanized, etc. objects). In some embodiments, the surface to which the adhesive device is applied may be a metallic alloy (e.g., bronze, brass, stainless steel, titanium-aluminum, titanium-aluminum-vanadium, cobalt-chromium, nickel-chrome, etc.), or may include an industrial material surface used in a marine, plumbing, earth-based (e.g., basalt, granite, limestone, sandstone, slate, etc.), paving (e.g., asphalt, concrete, cement), or piping setting. In some embodiments, the surface to which the adhesive composition is applied comprises a glass surface (e.g., glass (e.g., silica, aluminosilicate, borosilicate) or glazed objects).

In some embodiments, the surface to which the adhesive composition is applied may require or be related to a permanent application. In other embodiments, the surface to which the adhesive composition is applied may require or be related to a rapid or temporary application, for example, in the case of a leaking pipe or a leaking or sinking marine vessel (e.g., a boat). In some embodiments, the surface to which the adhesive composition is applied may require or be related to holding down and securing an object (e.g., tent, canopy, plant, tree, barrier, fence, net, dam, signage, appliance, boat, car, mobile home or camper) to the ground, pavement, or to any other device anchored to the ground that would otherwise separate and be mobile due to vibration, cyclic straining, or buoyancy forces (e.g. wave action, wake, wind or current flutter, floods), rain, hail, snow, sleet, or wind. Compositions disclosed herein may be applied to any such surface.

### Methods of Use of the Composition

Compositions comprising a therapeutic disclosed herein may be used in a variety of applications, for example, medical or dental applications. For example, adhesive compositions may be used in a wide variety of applications as disclosed herein. In an embodiment, the method of use comprises applying the composition, e.g., adhesive composition, to a site in need of repair or adhesion. In some embodiments, the method of use comprises applying pressure to one or more surfaces or obtrusions of a site for a predetermined period of time, for example, to hold a surface or object in place until the composition hardens.

In some embodiments, the method of use comprises using the adhesive composition for orthopedic and spinal indications; wherein the adhesive composition optionally comprising a therapeutic is applied to the surgery site, wherein the adhesive composition occludes access to the bone or bone wound surface to undesirable factors or wherein the therapeutic is either retained or locally and controllably released from the composition to treat any one of the following ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition mitigates post-operative pain. In some embodiments, the adhesive composition comprising a therapeutic can be used to join broken pieces of bone. In some embodiments, the composition comprising a therapeutic may be applied to any metal implants that required adhesion with the native bone or other preexisting metal implant.

In some embodiments, the composition comprising a therapeutic is used for hip surgery.

In some embodiments, the composition comprising a therapeutic is for knee surgery.

In some embodiments, the composition comprising a therapeutic is used for total ankle arthroplasty.

In some embodiments, the composition comprising a therapeutic is used for cranial flap fixation.

In some embodiments, the composition comprising a therapeutic is used in cases of osteosarcoma.

In some embodiments, the adhesive composition may be used for one or more of a hip surgery, knee surgery, total ankle arthroplasty, cranial flap fixation, or osteosarcoma.

In some embodiments, the method of use further comprises observation of a defect in an object (e.g., a wet, submerged, immersed, leaking, weeping, or oozing object) from which a fluid (e.g., an aqueous fluid) is emanating through a crack, fissure, breach or defect in the surface and a determination of the specific type of device (e.g., an adhesive device described herein) or the specific components thereof to utilize to seal or repair said object. In some embodiments, the adhesive composition optionally comprising a therapeutic may be applied to an object in a number of ways. For example, in some embodiments, the method of application comprises placement on an object that is wet, submerged, immersed, leaking, weeping, or oozing from the surface. Exemplary crack, fissures, breaches or defects in a surface include, but are not limited to, perforations, ruptures, pores, pits, tears, corrosions, erosions, abrasions, fractures, and the like.

In some embodiments, the method of use comprises application of the composition, e.g., adhesive composition, optionally comprising a therapeutic to a perforated object from which a flow of an aqueous medium (e.g., an aqueous medium described herein) emanates from the perforation. In such cases, the flow of the aqueous fluid may interact with the adhesive composition or components thereof, allowing the composition to solidify while in contact with the surface. In some embodiments, the adhesive composition occludes access to the object or object surface to undesirable factors and the therapeutic may be either retained or locally and controllably released from the composition. In some embodiments, the method further comprises observation of said interaction between the adhesive composition comprising a therapeutic and the aqueous medium. In some embodiments, the method comprises application of the adhesive composition optionally comprising a therapeutic to a wet surface, wherein the device and the aqueous medium interact, for example, resulting in the solidification and bonding of the adhesive composition to the surface. In some embodiments, the composition occludes access to the object or object surface to undesirable factors and the therapeutic may be either retained or locally and controllably released from the composition. In still other embodiments, the method comprises wetting the composition optionally comprising a therapeutic with an aqueous medium, followed by application of the composition to a surface, resulting in the solidification and bonding of the composition to the surface and wherein the composition occludes access to the object or object surface to undesirable factors and the therapeutic may be either retained or locally and controllably released from the composition. Exemplary perforated objects include, but are not limited to, pavement, bone, pipe, boat hull, pane of glass, boat deck, storage vessel, tank, or industrial process equipment. The perforated object may comprise metal or have a metal surface, wherein the metal surface is coated with chromium, nickel, zinc, tin, silver, or copper. These exemplary metal surfaces may also be coated through natural oxidation or corrosion processes to be titanium oxide, aluminum oxide, zinc oxide, chromium oxide, nickel oxide, tin oxide, silver oxide, iron oxide, or copper oxide. In any and all of these embodiments, the method may further comprise observation of said interaction between the adhesive composition comprising a therapeutic and the aqueous medium.

In other embodiments, the method further comprises use of an additional access device to aid in the application of the adhesive composition to a surface or region which is difficult to reach or to which minimally invasive approaches are desired. Exemplary additional access devices to aid in such application include, but are not limited to, an access tube, cannula, guide, or pipe. In some embodiments, the adhesive composition is injected or pushed through the additional access device.

### Kits

In some embodiments, different variants of the components of the adhesive composition may be packaged and marketed as a kit for specific applications.

In some embodiments, the kits may comprise a composition comprising: a multivalent metal salt (e.g., tetra-calcium phosphate); a compound of a Formula (e.g., Formula (I), Formula (II), Formula (III), Formula (IV), Formula (V), Formula (VI), or combinations thereof); optionally a therapeutic; optionally an additive; and an aqueous medium present together and sealed under good packaging practices to preserve the shelf life of the individual components. In some embodiments, the therapeutic and the aqueous medium may be packaged separately. In some embodiments, the composition, e.g., adhesive composition, may be present in a container. In some embodiments, preservation of the shelf life of the components within the kit includes a barrier to moisture ingress or maintenance of sterility. In some embodiments, wherein additives are included in said kit, they may be packaged within this container or within a separate container or compartment (e.g., multi-barrel syringe or multi-chambered cartridge or capsule). In some embodiments, the aqueous medium (e.g., water, solution, or suspension) may be provided in a separate container or compartment. In some embodiments, the kit may include additional components, e.g., devices, for the preparation or application of the composition, such as mixing bowls or surfaces, stirring sticks, spatulas, syringes, heat guns, agitators, triturators, applicator hand pieces, pumps, or other preparation or delivery devices. In some embodiments, the kit includes instructions for use, e.g., instructions for a method of using an adhesive composition, as disclosed herein.

### EXAMPLES

### Example 1: Exemplary Compounds

Listed on Table 1 are exemplary compounds that were found to produce self-setting adhesive compositions. Exemplary adhesive compositions were prepared by providing the compounds listed in Table 1 and tetra-calcium phosphate in a suitable receptacle and mixing with water to achieve the desired consistency. While water was used as the aqueous medium in the present compositions, the aqueous medium may instead be blood, saliva, serum, or a blood-based solution or suspension. In the present compositions, the solid components (compounds, tetra-calcium phosphate) were provided as particles. However, the solid components listed in the table may be provided in particle, granule, or fiber form, and the size of each of the components may vary as described in the Detailed Description. In some embodiments, the resulting properties, such as working and setting time, may be affected by these changes. The specific mean particle, granule, or fiber size for each solid component was selected to satisfy the use requirements as described in each of the embodiments.

Exemplary adhesive compositions were prepared comprising the compounds shown in Table 1. The compounds comprising glyphosine, O-Phospho-L-serine (OPLS), ethylenediaminetetraacetic acid (EDTA), propylenediamine tetraacetic acid, and aminotris(methylenephosphonic acid) (ATMP) were injectable, adhesive, tacky, and self-setting. After mixing, the compositions described were applied to the desired site and the adhesive properties examined, e.g., for tensile strength and durability.

TABLE 1
Exemplary Compounds

| Formula | Compound Name | Structure |
|---|---|---|
| I-a | N,N-Bis(phosphonomethyl)glycine (Glyphosine) | 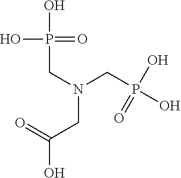 [SMILES: O=C(O)CN(CP(=O)(O)O)CP(=O)(O)O] |
| I-b | (Nitrilotris(methylene)) triphosphonic acid (ATMP) | 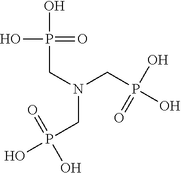 [SMILES: O=P(O)(O)CN(CP(=O)(O)O)CP(=O)(O)O] |
| II-a | Ethylenediamine tetraacetic acid (EDTA) | 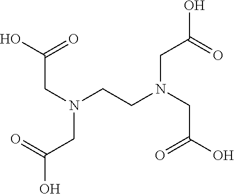 [SMILES: O=C(O)CN(CCN(CC(=O)O)CC(=O)O)CC(=O)O] |
| II-b | Egtazic acid (EGTA) | 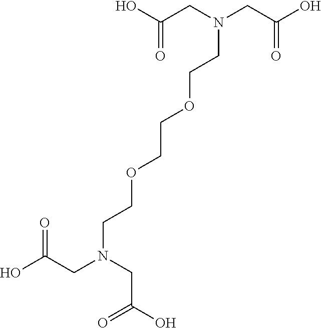 [SMILES: O=C(O)CN(CCOCCOCCN(CC(=O)O)CC(=O)O)CC(=O)O] |
| II-C | Propylenediaminetetraacetic acid (PDTA) | 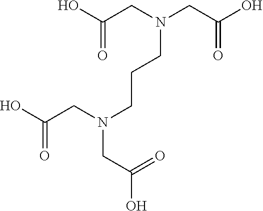 [SMILES: O=C(O)CN(CCCN(CC(=O)O)CC(=O)O)CC(=O)O] |
| III-a | Malonic Acid | 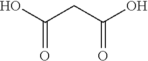 |
| III-b | Citric Acid | 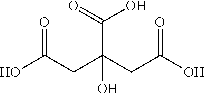 [SMILES: O=C(O)CC(O)(CC(=O)O)C(=O)O] |
| IV-a | O-Phospho-L-Serine (OPLS) | 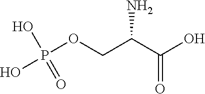 [SMILES: N[C@@H](COP(=O)(O)O)C(=O)O] |
| V-a | Phosphocreatine | 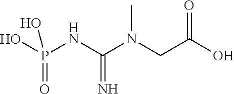 |
| VI-a | 2′-Deoxyadenosine monophosphate | 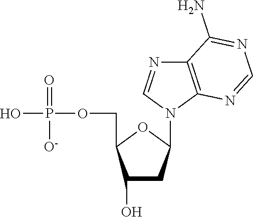 [SMILES: Nc1ncnc2c1ncn2[C@H]1C[C@@H](O)[C@@H](COP(=O)([O-])O)O1] |
| VI-b | 2′-Deoxyadenosine diphosphate | 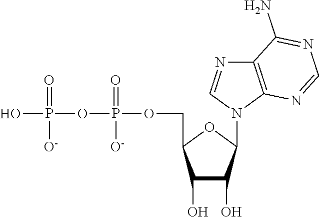 |

### Example 2. Therapeutic Retention and Release

In vitro studies described herein have shown that a therapeutic can be incorporated into the compositions and released or retained over time depending on the nature of therapeutic. The release profile of a target drug can be tailored to follow a specific time-dose curve using conventional time release technology such as passive diffusion.

### Example 2A. Sterilization of the Exemplary Therapeutics

Withstanding gamma sterilization is critical for a therapeutic to be included with the adhesive composition. Different therapeutics comprising pain relievers (e.g., locally acting analgesics (e.g., naproxen and diclofenac) or local anesthetics (e.g. bupivacaine and articaine) in powder form were gamma sterilized to check their stability.

### Method:

The therapeutics were dissolved in DMSO-D6 to prepare 5 mg/mL solutions. These solutions were transferred into a 5 mm NMR tube, and the NMR spectra were collected for pre-sterilization samples. The same therapeutics were then gamma sterilized 35-40 kGy and then dissolved in DMSO-D6 to prepare 5 mg/mL solutions. These solutions were transferred into a 5 mm NMR tube, and the NMR spectra were collected for post-sterilization data. Table 2 below compares the chemical shift in the therapeutics between pre and post gamma sterilization. The chemical shift data confirms that the therapeutics can withstand that gamma sterilization and does not undergo major chemical shift.

TABLE 2
Gamma Sterilization Chemical Shift

| | | δ (ppm) | |
|---|---|---|---|
| | | Post Gamma | Pre Gamma |
| Therapeutic | Hydrogen | Sterilization | Sterilization |
| Aspirin | 2 | 7.93 | 7.93 |
| | | 7.64 | 7.64 |
| | | 7.38 | 7.38 |
| | | 7.19 | 7.2 |
| | 1 | 2.24 | 2.24 |
| Ibuprofen | 7 | 12.24 | 12.25 |
| | 6 | 7.17 | 7.18 |
| | 6 | 7.09 | 7.1 |
| | 5 | 3.61 | 3.62 |
| | 4 | 2.4 | 2.4 |
| | 3 | 1.79 | 1.8 |
| | 2 | 1.32 | 1.33 |
| | 1 | 0.84 | 0.85 |
| Diclofenac | 2 | 10.42 | 10.41 |
| | 1 | 7.44 | 7.43 |
| | | 6.98 | 6.9 |
| | | 6.72 | 6.71 |
| | | 6.22 | 6.21 |
| Naproxen | 5 | 12.33 | 12.33 |
| | 4 | 7.78 | 7.77 |
| | | 7.71 | 7.7 |
| | | 7.4 | 7.39 |
| | | 7.29 | 7.28 |
| | | 7.15 | 7.14 |
| | 3 | 3.86 | 3.85 |
| | 2 | 3.8 | 3.79 |
| | 1 | 1.44 | 1.43 |
| Bupivacaine•HCl | 9 | 10.68 | 10.6 |
| | | 9.79 | 9.77 |
| | 8 | 7.12 | 7.12 |
| | 7 | 4.26 | 4.22 |
| | 7 | 3.51 | 3.5 |
| | 6 | 3.07 | 3.05 |
| | 5 | 2.31 | 2.3 |
| | 4 | 2.16 | 2.15 |
| | 3 | 1.68 | 1.67 |
| | 2 | 1.3 | 1.3 |
| | 1 | 0.9 | 0.89 |
| Lidocaine•HCl | 7 | 10.39 | 10.41 |
| | 6 | 9.9 | 9.91 |
| | 5 | 7.1 | 7.1 |
| | 4 | 4.24 | 4.23 |
| | 3 | 3.23 | 3.35 |
| | 2 | 2.17 | 2.19 |
| | 1 | 1.27 | 1.26 |
| Prilocaine | 10 | 10.26 | 10.21 |
| | 9 | 9.5 | 9.41 |
| | 8 | 8.98 | 8.97 |
| | 7 | 7.24 | 7.25 |
| | 6 | 4.16 | 4.14 |
| | 5 | 2.84 | 2.84 |
| | 4 | 2.21 | 2.21 |
| | 3 | 1.67 | 1.66 |
| | 2 | 1.55 | 1.54 |
| | 1 | 0.92 | 0.91 |
| Articaine | 11 | 10.58 | 10.55 |
| | 10 | 9.58 | n/a |
| | 9 | 9 | n/a |
| | 8 | 7.6 | 7.59 |
| | 7 | 4.13 | 4.12 |
| | 6 | 3.76 | 3.75 |
| | 5 | 2.85 | 2.84 |
| | 4 | 2.08 | 2.07 |
| | 3 | 1.68 | 1.65 |
| | 2 | 1.59 | 1.56 |
| | 1 | 0.92 | 0.91 |

### Example 2B. Affinity of Exemplary Therapeutics to Adhesive Compositions

Several formulations of compositions comprising pain relievers (e.g., locally acting analgesics (e.g., naproxen and diclofenac) or local anesthetics (e.g. bupivacaine and articaine) were investigated for their affinity to the adhesive composition. The affinity of the therapeutic depends on the nature of the therapeutic and its interaction with the composition. This is measured as the partition coefficient which is the ratio of the amount of drug released from the composition to the ratio of the amount of drug retained in the composition under specific conditions.

### Method:

The specific composition (e.g., adhesive composition) comprised 400 mg tetracalcium phosphate, 250 mg phosphoserine (a compound in FIG. 1V-a), and 135 μl of water that was prepared by mixing for 30 seconds, after which the mixture was formed into standard spherical substrates and allowed to cure. The substrate was then crushed using a mortar and pestle and used as a control. For the composition comprising a therapeutic, 1% w/w of the therapeutic was added to the tetracalcium phosphate and phosphoserine powder blend. The therapeutic was thoroughly mixed with tetracalcium phosphate and phosphoserine, after which deionized water was added and mixed for 30 seconds. Standard spherical substrates approximately 8 mm in diameter of adhesive composition were prepared. After curing, the substrate was crushed using mortar and pestle and the samples were incubated in 50 mL 1× PBS solution for 18 hours at 37° C. The partition coefficient of the therapeutic was evaluated using high-pressure liquid chromatography (HPLC) as shown in Table 3 below.

TABLE 3
Partition Coefficient

| Drug | Partition coefficient |
|---|---|
| Aspirin | 0.61 |
| Bupivacaine•HCl | 0.37 |
| Naproxen | 0.14 |
| Diclofenac•Na | 0.103 |
| Ibuprofen | 0 |
| Lidocaine | <0.0003 |
| Prilocaine | n/a |

### Example 2C. Release of Exemplary Therapeutics

Several formulations of compositions comprising pain relievers (e.g., locally acting analgesics (e.g., naproxen and diclofenac) or local anesthetics (e.g. bupivacaine and articaine) were investigated. The retention of the pain reliever within the adhesive composition may aid in the mitigation of post-operative surgical pain. The release study was analyzed using elution studies, wherein HPLC was used to analyze the amount of pain reliever released over time. The elution profile of the drugs listed above as from an adhesive composition matrix was analyzed over a 7-day period.

### Method:

The specific composition (e.g., adhesive composition) comprised 400 mg tetracalcium phosphate, 250 mg phosphoserine (a compound in Table 1), and 135 μl of water that was prepared by mixing for 30 seconds, after which the mixture was loaded into a syringe and injected out to form standard spherical substrates and allowed to cure. This formulation was used as a control. For the composition comprising a therapeutic, a predetermined amount of therapeutic by weight %, was thoroughly mixed with tetracalcium phosphate and phosphoserine, after which distilled water was added and mixed for 30 seconds. Two formulations per drug were tested by adding 2.5% w/w and 10% w/w to the tetracalcium phosphate and phosphoserine powder blend. Standard spherical substrates of approximately 8 mm in diameter of adhesive compositions were prepared. After curing, the samples were incubated in 50 mL 1× PBS solution for 7-days at 37° C. The release profile of the drugs was evaluated using high-pressure liquid chromatography (HPLC) as shown in FIG. 24.

### Example 2D. Release of Exemplary Therapeutics from Porous Adhesive Composition Formulation

Several formulations of compositions comprising pain relievers (e.g., local anesthetics (e.g. bupivacaine)) were investigated. The retention of the pain reliever within the porous adhesive composition may avoid or mitigate post-operative surgical pain. The release study was analyzed using elution studies, where HPLC was used to analyze the amount of pain reliever released over time. The elution profile of bupivacaine from a porous adhesive composition matrix was analyzed over a 7-day period.

### Method:

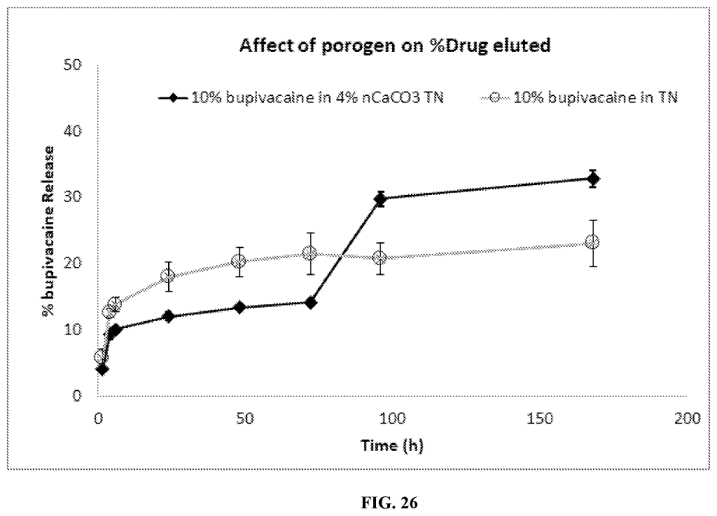

The specific composition (e.g., adhesive composition) comprised 400 mg tetracalcium phosphate, 250 mg phosphoserine, 26 mg of calcium carbonate and 135 μl of water and was prepared by mixing for 30 seconds, after which the mixture was loaded into a syringe and injected out to form standard spherical substrates and allowed to cure. The particle size of calcium carbonate was between 15 nm to 40 nm. This formulation was used a control for the study. The control showed porosity of a control substrate was about 18.5% when measured using mercury intrusion porosimetry. For the adhesive compositions comprising a therapeutic, a predetermined amount of therapeutic by weight %, was thoroughly mixed with tetracalcium phosphate and phosphoserine, after which distilled water was added and mixed for 30 seconds. A bupivacaine formulation was tested by adding 10% w/w of the drug to the tetracalcium phosphate and phosphoserine powder blend. Standard spherical substrates of approximately 8 mm in diameter of porous adhesive bone regenerative were prepared. After curing, the samples were incubated in 50 mL 1× PBS solution for 7-days at 37° C. The release profile of the drugs was evaluated using HPLC as shown in FIG. 26.

### Example 2E. Chemical Interactions Between Adhesive Composition Matrices and Exemplary Therapeutics

Several formulations of compositions comprising pain relievers (e.g., locally acting analgesics (e.g., naproxen or diclofenac) or local anesthetics (e.g. bupivacaine and articaine)) were investigated. The retention of the pain reliever within the adhesive composition may avoid or mitigate post-operative surgical pain. This study was used to evaluate whether there is any chemical interaction of the drug with the adhesive composition matrix and was analyzed using ssNMR to study the amount of pain reliever released over time. Particularly, the interaction of naproxen in the adhesive composition matrix was analyzed after 4 days.

### Method:

The specific compositions (e.g., adhesive compositions) comprised 400 mg tetracalcium phosphate, 250 mg phosphoserine (a compound in FIG. 1V-a), and 130 μl of water was prepared by mixing for 30 seconds, after which the mixture was loaded into a syringe and injected out to form standard spherical substrates and allowed to cure. For the adhesive compositions comprising a therapeutic, a predetermined amount of therapeutic by weight %, was thoroughly mixed with tetracalcium phosphate and phosphoserine, to which distilled water was added and mixed for 30 seconds. A formulation was tested by adding 8% w/w naproxen to the tetracalcium phosphate and phosphoserine blend by weight. Standard spherical substrates of approximately 0.6 g of adhesive composition were prepared with 8% naproxen. After curing, the samples were crushed and incubated in 10 mL 1× PBS solution for 4-days at 37° C. The interaction of naproxen was evaluated using ssNMR as shown in FIG. 27 below.

### Example 3. Retention of Exemplary Therapeutics

Several formulations of compositions comprising antibiotics (AB) were investigated. The retention of the AB within the compositions may avoid or prevent bacterial colonization on or within the cured adhesive composition or any post-operative infection that could develop. The retention study was analyzed using elution studies, wherein high-pressure liquid chromatography (HPLC) was used to analyze the amount of AB released over time. Particularly, the elution profile of gentamicin from adhesive composition matrix was analyzed at days 1, 3, 5 and 10.

### Method:

The specific composition (e.g., adhesive composition) comprised 400 mg tetracalcium phosphate, 250 mg phosphoserine (Compound IV-a in Table 1), and 130 μl of water and was prepared by mixing for 30 seconds, after which the mixture was formed into a cylindrical plug and allowed to cure. For the adhesive compositions comprising a therapeutic, a predetermined amount of therapeutic by weight %, was thoroughly mixed with tetracalcium phosphate and phosphoserine, to which distilled water was added and mixed for 30 seconds, after which the mixture was formed into a cylindrical plug and allowed to cure. Several compositions were tested by adding either 2.5% w/w or 5% w/w of gentamicin to the tetracalcium phosphate and phosphoserine powder blend. Each cylinder was immersed in a polypropylene tube with 5 ml phosphate buffered saline (PBS) and shaken in a rotator at 37° C. Over the study period of 10 days, daily transfer of the cement cylinder into a test tube with PBS was conducted after saline washings. Elution samples of PBS was collected at various time points over a period of 10 days. The concentration of gentamicin was determined using high-performance liquid chromatography (HPLC) as shown in FIG. 24. The control was adhesive composition with no gentamicin, and the standard was different concentration of gentamicin alone. The elution results shown in FIG. 24 confirm that most of gentamicin was retained in the adhesive composition after 10 days incubation.

1. A composition comprising a multivalent metal salt, a compound of a Formula (described herein) selected from Formula I, Formula II, Formula III, Formula IV, Formula V, and Formula VI, a therapeutic, and an aqueous medium.

2. The composition of claim 1, wherein the multivalent metal salt is a salt of an alkaline earth element.

3. The composition of claim 2, wherein the multivalent metal salt comprises calcium.

4. The composition of claim 3, wherein the multivalent metal salt comprises tetracalcium phosphate or tricalcium phosphate (e.g., alpha tricalcium phosphate or beta tricalcium phosphate).

5. The composition of claim 1, wherein the compound of Formula (I) comprises: 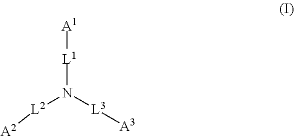
or a salt thereof, wherein each of A¹, A², and A³ is independently selected from an acidic group (e.g., a carboxyl or phosphonyl); and each of L¹, L², and L³ is independently bond, alkylene (e.g., C₁-C₆ alkylene), or heteroalkylene (e.g., C₁-C₆ heteroalkylene).

6. The composition of claim 5, wherein the compound of Formula (I) comprises a compound in Table 1, e.g., glyphosine or ATMP.

7. The composition of claim 1, wherein the compound of Formula (II) comprises: 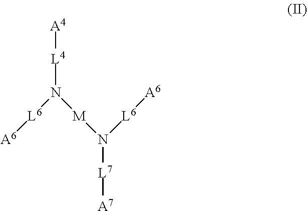
or a salt thereof, wherein each of A⁴, A⁵, and A⁶, is independently selected from an acidic group (e.g., a carboxyl or phosphonyl); A⁷ is selected from an acidic group (e.g., a carboxyl or phosphonyl), a hydrogen atom, an alkyl, an aryl, a hydroxy group, a thio group, and an amino group; each of L⁴, L⁵, L⁶, and L⁷ is independently bond, alkylene (e.g., C₁-C₆ alkylene), or heteroalkylene (e.g., C₁-C₆ heteroalkylene); and M is alkylene (e.g., C₁-C₆ alkylene) or heteroalkylene (e.g., C₁-C₆ heteroalkylene).

8. The composition of claim 7, wherein the compound of Formula (II) comprises a compound in Table 1, e.g., EDTA, EGTA, or PDTA.

9. The composition of claim 1, wherein the compound of Formula (III) comprises: 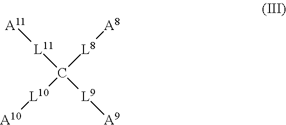
or a salt thereof, wherein each of A⁸ and A⁹ is independently selected from an acidic group (e.g., a carboxyl or phosphonyl); each of A¹⁰ and A¹¹ is independently selected from an acidic group (e.g., a carboxyl or phosphonyl), a hydrogen atom, an alkyl, aryl, a hydroxy group, a thio group, and an amino group; each of L⁸, L⁹, L¹⁰ and L¹¹ is independently bond, alkylene (e.g., C₁-C₆ alkylene), or heteroalkylene (e.g., C₁-C₆ heteroalkylene).

10. The composition of claim 9, wherein the compound of Formula (III) comprises a compound in Table 1, e.g., malonic acid or citric acid.

11. The composition of claim 1, wherein the compound of Formula (IV) comprises: 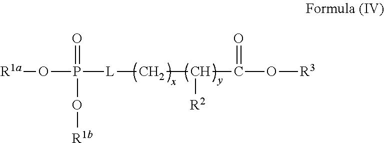
or a salt thereof, wherein L is O, S, NH, or CH₂; each of R^{1a} and R^{1b} is independently H, an optionally substituted alkyl, or an optionally substituted aryl; R² is H, NR^{4a}R^{4b}, C(O)R⁵, or C(O)OR⁵; R³ is H, an optionally substituted alkyl, or an optionally substituted aryl; each of R^{4a} and R^{4b} is independently H, C(O)R⁶, or an optionally substituted alkyl; R⁵ is H, an optionally substituted alkyl, or an optionally substituted aryl; R⁶ is an optionally substituted alkyl or an optionally substituted aryl; and each of x and y is independently 0, 1, 2, or 3.

12. The composition of claim 11, wherein the compound of Formula (IV) comprises a compound in Table 1, e.g., phosphoserine.

13. The composition of claim 1, wherein the compound of Formula (V) comprises: 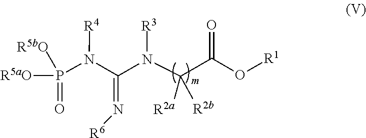
or a salt thereof wherein R¹ is H, optionally substituted alkyl, optionally substituted alkenyl, optionally substituted alkynyl, optionally substituted aryl, or optionally substituted heteroaryl; each of R^{2a} and R^{2b} is independently H, optionally substituted alkyl, hydroxy, alkoxy, or halo; each of R³ and R⁴ is independently H or optionally substituted alkyl; each of R^{5a} and R^{5b} is independently H, optionally substituted alkyl, optionally substituted alkenyl, optionally substituted alkynyl, optionally substituted aryl, or optionally substituted heteroaryl; R⁶ is H or optionally substituted alkyl; and m is 1, 2, 3, 4, or 5.

14. The composition of claim 13, wherein the compound of Formula (V) comprises a compound in Table 1, e.g., phosphocreatine.

15. The composition of claim 1, wherein the compound of Formula (VI) comprises: 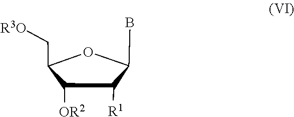
or a salt thereof, wherein B is a nucleobase; R¹ is H, OR⁴, or halo; R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl; R³ is H, optionally substituted alkyl, or a phosphate moiety (e.g., monophosphate or diphosphate); and R⁴ is H, optionally substituted alkyl, optionally substituted alkenyl, optionally substituted aryl, optionally substituted heteroaryl, optionally substituted cycloalkyl, or optionally substituted heterocyclyl.

16. The composition of claim 15, wherein the compound of Formula (VI) comprises a compound in Table 1, e.g., 2′-deoxyadenosine monophosphate or 2′-deoxyadenosine diphosphate.

17. The composition of claim 1, wherein the aqueous medium is water.

18. The composition of claim 1, wherein the compound of the Formula (e.g., a compound of any one of Formulas (I), (II), (III), (IV), (V), or (VI), or a salt thereof) is present in an amount from about 10% to about 90% weight by weight (w/w) of the total composition.

19. The composition of claim 1, wherein the therapeutic comprises a pain reliever, an antibiotic, chemotherapeutic, hormone, protein, or contrast agent.

20. The composition of claim 1, wherein the therapeutic comprises a compound in Table 2.

21. The composition of claim 1, wherein the therapeutic is present in an amount from about 0.1% to about 40% weight by weight (w/w) of the total composition.

22. The composition of claim 1, wherein the composition has an adhesive strength upon curing of greater than 100 kPa.

23. The composition of claim 1, further comprising an additive.

24. The composition of claim 23, wherein the additive is a salt (e.g., calcium carbonate, calcium bicarbonate, sodium carbonate, or sodium bicarbonate), a filler, a formulation base, an abrasive, a coloring agent, a flavoring agent, a polymer, a viscosity modifier, or an antimicrobial agent.

25. The composition of claim 23, wherein the additive is a solidified form of the composition.

26. The composition of claim 24, wherein the polymer is present in the form of a powder, granule, or fiber.

27. The composition of claim 1, wherein the multivalent metal salt, the compound of the Formula selected, and the therapeutic (e.g., the dry components) of the composition are present in the form of a powder or granule.

28. The composition of any one of claims 26 or 27, wherein the mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.

29. The composition of any one of claims 26 or 27, wherein the particle size distribution is multi-modal to include any combination of mean particle sizes.

30. The composition of claim 23, wherein the additive is a solidified form of the composition in the form of porous granules.

31. The composition of claim 30, wherein the granules are supplied with a various proportion of porosity and various sized internal pores.

32. The composition of claim 31, wherein, the pores communicate with the granule surface.

33. The composition of claim 1, wherein the composition is porous.

34. The composition of any one of claims 31-33, wherein the pores are formed when a salt additive reacts with the dry components of the composition and releases carbon dioxide gas causing the composition to expand and leaving pores once the CO₂ gas has dissipated.

35. A method of forming a composition comprising a multivalent metal salt, a compound of any one of Formulas (I), (II), (III), (IV), (V), or (VI), or a salt thereof, and a therapeutic.

36. The method of claim 35, wherein at least one of the multivalent metal salt, the compound of any one of Formulas (I), (II), (III), (IV), (V), or (VI), or a salt thereof, and the therapeutic are present within the composition as porous granules.

37. The method of claim 36, wherein the method of creating said porous granules comprises:
a. the inclusion of a salt additive reacting with one of the multivalent metal salt, the compound of the Formula selected, or the therapeutic; and
b. once the composition has solidified into its cement state, grinding the composition (e.g., by jaw crusher, co-mill, jet-mill, or ball-mill); and optionally
c. sieving the resultant ground up cement to control a preferable size range.

38. The composition of any one of claims 30-34 or the method of any one of claims 35-37, wherein the pores range in size from 0.01 mm to 1.0 mm.

39. The composition of any one of claims 30-34 or the method of any one of claims 35-37, wherein porosity is determined by the amount of salt present in the composition or granule.

40. The composition or method of claim 39, wherein the amount of salt additive ranges from 0.5% to 20% w/w volume of the solid components of the composition.

41. The composition or method of claim 40, wherein the resulting porosity ranges from 5% to 95%.

42. The composition of claim 1 or method of claim 35, wherein the therapeutic is a pain reliever, an antibiotic, chemotherapeutic, hormone, protein, or contrast agent.

43. The composition or method of claim 42, wherein the pain reliever is an opioid, non-steroidal anti-inflammatory (NSAID), local anesthetic, or gabapentinoid.

44. The composition or method of claim 42, wherein the antibiotic is penicillin (β-lactams), cephalosporins (γ-lactams), macrolides, lincomycin, nitroimidazoles, carbapenems, fluoroquinolones, sulfonamides, tetracyclines, aminoglycosides, quinolone, polyketides, or glycopeptides.

45. The composition or method of claim 42, wherein the chemotherapeutic is alkylating agents, antimetabolites, antibiotics, topoisomerase, or tyrosine kinase inhibitors.

46. The composition or method of claim 42, wherein the hormone is a growth hormone or cholecalciferol.

47. The composition or method of claim 42, wherein the contrast agent is gadolinium-based, barium-based, or iron/iron oxide-based.

48. The composition of claim 1 or method of claim 35, wherein the therapeutic is released from the composition.

49. The composition or method of claim 48, wherein the release rate of the therapeutic is determined by the porosity of the composition.

50. The composition or method of claim 48, wherein the release rate of the therapeutic is determined by the rate at which the composition biodegrades.

51. The composition or method of claim 48, wherein the release rate of the therapeutic is determined by the partition coefficient between the therapeutic and the adhesive composition.

52. The composition of claim 1 or method of claim 35, wherein the composition is bone regenerative, e.g., resorbable by native bone and replaced with bone maintaining volume.

53. The composition of claim 1 or method of claim 35, wherein the composition is utilized to adhere bone to bone, to adhere a device, e.g., a rod or a plate, to bone, to adhere one device or structure to another, or to fill a bone void.

54. A kit comprising an adhesive composition comprising a multivalent metal salt; a compound of Formula (I), Formula (II), Formula (III), Formula (IV), Formula (V), Formula (VI), a salt thereof, or a combination thereof; a therapeutic; and an aqueous medium.

55. The kit of claim 54, further comprising a device for preparation of the adhesive composition or a device for application of the adhesive composition.

56. The kit of claim 54, wherein said device and adhesive composition can undergo sterilization, e.g., gamma irradiation, without compromising the chemical structure of the therapeutic.

57. A method of using composition of any one of claims 1-34, comprising the steps of
a) mixing and preparing components of the composition; and
b) applying the composition to a surgery site.

58. The method of claim 57, wherein said composition is utilized to fill bone voids, specifically dental voids (e.g., voids from wisdom tooth removal).

59. The method of claim 57, wherein said composition comprises a pain reliever to treat pain.

60. The method of claim 57, wherein said composition is utilized in bone to bone fracture fixation.

61. The method of claim 57, wherein said composition comprises an antibiotic to prevent infection.
